(19) United States
(12) Patent Application Publication
(10) Pub. No.: US 2026/0079167 A1
(43) Pub. Date: Mar. 19, 2026

(54) BIOMEDICAL TEST FOR AUTISM

(71) Applicants: ARIZONA BOARD OF REGENTS ON BEHALF OF ARIZONA STATE UNIVERSITY, Tempe, AZ (US); RENSSELAER POLYTECHNIC INSTITUTE, Troy, NY (US)

(72) Inventors: James Adams, Tempe, AZ (US); Rosa Krajmalnik-Brown, Chandler, AZ (US); Juergen Hahn, Troy, NY (US); Fatir Qureshi, Troy, NY (US)

(21) Appl. No.: 18/871,063

(22) PCT Filed: Jun. 1, 2023
(86) PCT No.: PCT/US2023/024190

§ 371 Date: Dec. 2, 2024

Related U.S. Application Data
(60) Provisional application No. 63/347,874, filed on Jun. 1, 2022.

(51) Int. Cl. G01N 33/68; G01N 33/84

(52) U.S. Cl. CPC G01N 33/6896; G01N 33/84; G01N 2570/00; G01N 2800/28; G01N 2800/52

(57) ABSTRACT

Diagnostic metabolites and methods of using the metabolites for the diagnosis of autism spectrum disorder (ASD) are provided. The metabolites comprise a collection of 47 metabolites that were significantly different between the autistic and typically developing groups, and combinations of two or more of the metabolites are used to accurately diagnose ASD.

### CROSS REFERENCE TO RELATED APPLICATIONS

The present application claims the benefit of U.S. Provisional Patent Application No. 63/347,874, entitled, “DEVELOPMENT OF A BIOMEDICAL DIAGNOSTIC TEST FOR AUTISM” filed Jun. 1, 2022. The content of the aforementioned application is hereby incorporated by reference in its entirety.

### FIELD OF THE TECHNOLOGY

The invention relates to methods for identifying subjects with autism spectrum disorder.

### BACKGROUND

Autism spectrum disorder (ASD) is a neurodevelopmental condition which is estimated to affect about 1 in 44 children in the United States. This condition is defined by difficulty in communication, social interaction, and restricted repetitive behaviors. Despite being categorized and diagnosed by a set of behavioral criteria, ASD is known to be associated with several co-occurring conditions that affect a multitude of physiological systems. As ASD etiology is understood to be a consequence of environmental and genetic factors, identifying distinctive metabolomics profiles of individuals with ASD has been a frequent subject of investigation.

ASD is a complex disorder which can be difficult to diagnose, and the current diagnosis is based on an assessment of social communication and behavior. Currently there is no widely accepted medical test for the diagnosis of autism, although several have been proposed. A number of metabolomic differences have been observed in individuals with ASD, many of which have also been examined for their potential role in this condition's clinical pathology. Differences in mitochondrial metabolism, the gastrointestinal system and redox regulation have been associated to varying degrees with ASD. Divergences in metabolite profiles between children with ASD and their typically developing cohorts have been shown to exhibit significant differences up to the point where predictions about which metabolic profiles belong to the ASD or TD group have been made. Furthermore, modulating metabolomic pathways holds significant promise as the basis to develop therapies addressing ASD co-occurring conditions and symptoms.

### SUMMARY

In some aspects, provided herein is a method of identifying Autism Spectrum Disorder (ASD) in a subject suspected of having or at risk of having ASD, the method comprising: measuring levels of a combination of two or more metabolites selected from the metabolites listed in Table 1 in a biological sample obtained from the subject; (b) applying the measured levels of each metabolite against a control panel of metabolite levels created by measuring metabolite levels of the one or combination of metabolites in control typically developing (TD) subjects, wherein the applying comprises: (i) calculating the sensitivity (TPR) and specificity (TNR) for the combination of metabolites using Fisher Discriminant Analysis (FDA) or support vector machines (SVM); and (ii) calculating the area under the curve (AUC) of the receiver operating characteristic (ROC) curve (AUROC) for the combination of two or more metabolites using the FPR and TPR calculated in (b); and (c) indicating ASD is present if the levels of each of the two or more metabolites in the biological sample are significantly different from the levels of the two or more metabolites in the control panel of metabolite levels if the AUROC value for the combination of metabolites is about 0.85 or higher.

In some aspects of the method, the metabolite is a metabolite associated with the one or combination of two or more metabolites identified as having a level in the biological sample significantly different from the level of the one or combination of metabolites in the control sample. In some aspects, the metabolites listed in Table 1 are identified by comparing the measured level of the metabolite in the biological sample to the level of the metabolite in the control panel of metabolite levels using a univariate statistical analysis method selected from hypothesis testing, evaluating the area under the receiver operator curve (AUROC) values, or a combination of both.

In some aspects, hypotheses testing is performed by evaluating the type of distribution for each of the ASD and TD groups' measurements and selecting an appropriate parametric or non-parametric test. In some aspects, a parametric or non-parametric test is selected by determining the normality and variance of each individual clinical measurement variable for both the ASD and TD groups separately. In some aspects, a parametric test is performed if a normality assumption holds true for the ASD and TD groups. In some aspects, an equal variance t-test or Welch's test (unequal variance t-test) is performed if the observed variance is significantly different between the ASD and TD groups. In some aspects, a Mann-Whitney test is performed if the ASD and TD groups follow the same non-parametric distribution.

In further aspects, the ASD and TD groups are adjusted by their means and subjected to the Kolmogorov-Smirnov test where different distributions are observed. In some aspects, the method further comprises determining the false discovery rate (FDR) for each measurement using a leave-one-out (L-1-O) approach.

In some aspects, the combination of two or more metabolites are identified by performing multivariate analysis using Fisher Discriminant Analysis (FDA) and support vector machines (SVM) and selecting the combinations of two or more metabolites comprising the best FDA or SVM measure for each combination of elements. In some aspects, performing FDA comprises: (a) evaluating all possible combinations of two, three, and four-metabolites from among the metabolites of Table 1; (b) examining the fitted AUROC and performance when subjected to cross-validation using leave-one-out cross-validation; and (c) calculating the area under the AUROC value for each combination of two, three, and four-metabolites; wherein the combination of two or more metabolites comprises the combinations of two, three, and four-metabolites comprising the highest 1000 AUROC values following leave-one-out cross-validation.

In some aspects, the method further comprises using a greedy algorithm to identify combinations of metabolites comprising five or more metabolites. In some aspects, performing SVM comprises assessing all possible combinations of 5-metabolites and subjecting each combination of five or more metabolites leave-one-out cross-validation if the combination of five or more metabolites attains an accuracy greater than 0.90.

In some aspects, the biological sample is whole blood, plasma, red blood cells (RBCs), urine, or any combination thereof. In some aspects, the combination of two or more metabolites comprises a combination of metabolites of Table 7 and Table 2. In some aspects, the control panel of metabolite levels is stored on a computer system.

In some aspects, the method identifies the presence of ASD at birth or pre-birth. In some aspects, the levels of a combination of two metabolites are measured and analyzed. In some aspects, the combination of two metabolites is selected from the combinations of two metabolites listed in Table 7. In some aspects, the levels of a combination of three metabolites are measured. In some aspects, the levels of the three metabolites are the levels of free sulfate in plasma, the level of uridine in plasma, and the level of beta-amino isobutyrate in plasma. In some aspects, the levels of a combination of four metabolites are measured. In some aspects, the levels of the four metabolites are the level of free sulfate in plasma, the level of uridine in plasma, the level of homo cystine in plasma, and the level of beta-amino isobutyrate in plasma. In some aspects, the levels of a combination of five metabolites are measured. In some aspects, the combinations of five metabolites are the levels of the combinations of five metabolites listed in Table 2. In some aspects, the levels of the five metabolites are the level of free sulfate in plasma, the level of uridine in plasma, the level of initial homo cystine in plasma, the level of beta-amino isobutyrate in plasma, and the level of magnesium in the serum. In some aspects, the levels of the five metabolites are the level of free sulfate in plasma, the level of uridine in plasma, the level of homo cystine in plasma, the level of beta-amino isobutyrate in plasma, and the level of tryptophan in the plasma. In some aspects, the levels of the five metabolites are the level of free sulfate in plasma, the level of magnesium in the serum, the level of homo cystine in plasma, the level of uridine in plasma, and the level of beta-amino isobutyrate in plasma. In some aspects, the levels of a combination of six metabolites are measured. In some aspects, the levels of the five metabolites are the level of free sulfate in plasma, the level of homo cystine in plasma, the level of uridine in plasma, the level of beta-amino isobutyrate in plasma, the level of magnesium in the serum, and the level of copper in RBCs.

In some aspects, the method further comprises assigning a medical, behavioral, and/or nutritional treatment protocol to the subject based on metabolites in the biological sample that are significantly different from the level of the metabolite in the control panel of metabolite levels if the AUROC value for the combination of metabolites is about 0.85 or higher. In some aspects, the treatment protocol comprises adjusting the level of one or a combination of two or more metabolites in the subject. In some aspects, the treatment protocol comprises administering a combination of anti-oxidants and a source of sulfate.

In some aspects, the disclosure encompasses a method of determining a personalized treatment protocol for a subject suspected of having or at risk of having ASD, the method comprising identifying ASD in a subject suspected of having or at risk of having ASD using a method disclosed herein, and assigning a personalized medical, behavioral, or nutritional treatment protocol to the subject if the subject is determined to have ASD. In some aspects, the treatment protocol comprises administering a combination of anti-oxidants and a source of sulfate.

In some aspects, provided herein is a method of monitoring a therapeutic effect of an ASD treatment protocol in a subject suspected of having or at risk of having ASD, the method comprising: (a) measuring in a first biological sample obtained from the subject the level of one or a combination of two or more metabolites selected from the metabolites listed in Table 1 and any combination thereof; (b) measuring in a second biological sample obtained from the subject at a period of time after the first biological sample is obtained the level of the one or combination of two or more metabolites; and (c) comparing the level of the one or combination of two or more metabolites in the first sample and the second sample; wherein maintenance of the level of the one or combination of two or more metabolites or a change of the level of the one or combination of two or more metabolites to a level of the one or combination of two or more metabolites in a control panel of metabolite levels created by measuring metabolite levels of the one or combination of two or more metabolites in control TD subjects is indicative that the treatment protocol is therapeutically effective in the subject.

Further provided herein is a kit for identifying Autism Spectrum Disorder (ASD) in a subject suspected of having or at risk of having ASD, determining a personalized treatment protocol, monitoring a therapeutic effect of an ASD treatment protocol, or any combination thereof, the kit comprising: (a) a container for collecting a biological sample from the subject; (b) solutions and solvents for preparing an extract from a biological sample obtained from the subject; and (c) instructions for (i) preparing the extract, (ii) measuring the level of one or more metabolites selected from the metabolites listed in Table 1; and (iii) applying the measured metabolite levels against a control panel of metabolite levels obtained from typically developing (TD) individuals.

### BRIEF DESCRIPTION OF THE FIGURES

FIG. 1 is a correlation network plot of correlation between significant biochemical and xenobiotic compounds in the TD cohort (Strength of correlation is visualized by line thickness, positive correlations are in blue and negative correlations are in red). In order for a relationship to be deemed significant the correlation coefficient was selected to be greater than 0.35, FDR less than 0.10 and p-value less than 0.05. In total, 378 significant correlations were observed that met these criteria. NADP and total sulfate had the greatest number of relationships, with 19 significant relationships. Correlation pairs with 3 or fewer connected nodes were removed, and only those relationships with r>0.40 are presented (see details in FIG. 9).

FIG. 2 is a correlation network plot of correlation between significant biochemical and xenobiotic compounds in the ASD cohort (strength of correlation is visualized by line thickness, positive correlations are in blue and negative correlations are in red). In order for a relationship to be deemed significant the correlation coefficient was selected to be greater than 0.35, FDR less than 0.10 and p-value less than 0.05. In total, 212 significant correlations (106 pairs) were observed. Acetylcholine had the greatest number of relationships, with 14 significant relationships. Correlation pairs with 3 or fewer connected nodes were removed (see details in FIG. 10).

FIG. 3 is a plot of the marker prevalence among the top 1000 FDA 5-marker models as judged by their performance on the test set. Among the prominent potential biomarkers are free sulfate, uridine and beta-amino isobutyrate (highlighted in black). Each of these were present in more than 75% of the top models. Free sulfate was present in every single top model.

FIG. 4 is a plot of the marker prevalence among the top 1000 4-marker FDA models as judged by their performance on the test set, with both total and free sulfate excluded. Due to the predominance of sulfate in model panels, models with other constituents were explored by conducting the FDA analysis with these two metabolites excluded. The metabolites observed to be most prevalent in the resulting models were highlighted in light grey and include (A) glutathione present in 43.3% (B) uridine present in 74.7% and (C) homocystine+homocysteine present in 32.1% models.

FIG. 5 is a plot of the marker prevalence among the top 1000 5-marker SVM models as judged by their performance on the test set. Among the most prominent potential biomarkers are (A) free sulfate in serum, (B) uridine (C) tryptophan (D) beta-amino isobutyrate and (E) Copper in whole blood.

FIG. 6 is a plot of the univariate distribution for free sulfate in plasma, which was the metabolite that had the highest AUROC (0.90).

FIG. 7A is the histogram of the FDA Scores for the 6-marker optimized model based upon cross-validated AUROC value.

FIG. 7B is the histogram of the FDA Scores for the 5-marker optimized model based upon cross-validated AUROC value.

FIG. 8 is a boxplot of the FDA Scores for both the 5-marker and 6-marker optimized model based upon cross-validated AUROC value. Each box represents scores that fall between the 25th-75th percentile for that respective set of scores.

FIG. 9 is a correlation network between significant biochemical and xenobiotic compounds in the TD cohort (strength of correlation is visualized by line thickness, positive correlations are in blue and negative correlations are in red). In order for a relationship to be deemed significant the correlation coefficient had to be greater than 0.35, FDR less than 0.10 and p-value less than 0.05.

FIG. 10 is a correlation network between significant biochemical and xenobiotic compounds in the ASD cohort (strength of correlation is visualized by line thickness, positive correlations are in blue and negative correlations are in red). For a relationship to be deemed significant the correlation coefficient had to be greater than 0.35, FDR less than 0.10 and p-value less than 0.05.

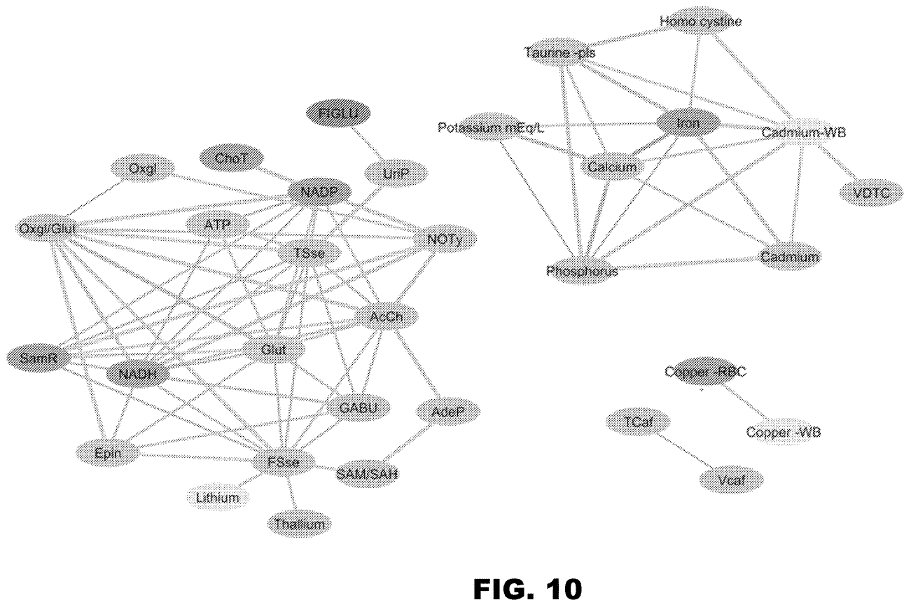

### DETAILED DESCRIPTION

Autism Spectrum Disorder (ASD) is currently primarily diagnosed using cognitive and behavioral testing. Such testing is subjective, and usually not discriminative until subjects are at least 2-3 years of age. Consequently, a diagnosis of ASD is typically not made until even later around 4-5 years of age. The average age of diagnosis in the US is approximately 4.5 years. Thus, patient identification often crucially lags when earlier intervention is important to mitigating the symptoms of ASD. The present disclosure is based at least in part on the discovery of a method that makes it possible to identify subjects at earlier age (i.e., earlier than about 2 years of age) as having or having high risk of ASD. Specifically, the methods disclosed herein examine 47 metabolites. The inventors have shown that certain metabolites, when analyzed as described herein provide a strong signal differentiating autistic and neurotypical development groups. Thus the inventors have provided a medical test that can be used for identifying ASD in a subject. The test for ASD disclosed herein has high sensitivity and specificity, allowing earlier screening and identification of individuals with autism. Furthermore, the tests will provide valuable guidance for personalizing treatments and evaluating treatment efficacy.

Among the combinations of metabolites disclosed in this invention, when analyzed as described herein, result in an unusually high accuracy (sensitivity and specificity). The high accuracy is attainable with analysis of as few as 2 of the metabolites, and that accuracy can be improved on by including more metabolites in the analysis. Cross-validation with leave-one-out methods is used to increase test confidence. Further, the biomarkers identified in the tests can be used to personalize ASD treatments for an individual, and to determine if a treatment is effective for that individual. For example, oxidative stress if found to be high and sulfation low, then this indicates that a treatment combination of anti-oxidants and sources of sulfate would be beneficial, and remeasuring and reanalyzing those biomarkers after treatment will reveal if the dosage is sufficient to regain metabolic balance.

### I. Terminology

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein “Autism spectrum disorder” or “ASD” is a complex neurodevelopmental condition characterized by widespread abnormalities of social interactions and communication, as well as restricted interests and repetitive behaviors. ASD typically appears during the first three years of life and manifests in characteristic symptoms or behavioral traits. A diagnosis of ASD now includes several conditions that used to be diagnosed separately: autistic disorder, pervasive developmental disorder not otherwise specified (PDD-NOS), and Asperger syndrome. All of these conditions are now encompassed by the diagnostic criteria for autism spectrum disorder as set forth in the American Psychiatric Association's Diagnostic & Statistical Manual of Mental Disorders, Fifth Edition (DSM-V).

In addition to the spectrum of symptoms seen within these principal diagnostic criteria, ASD individuals display a wide range of neurological comorbidities, including intellectual disability, epilepsy, and anxiety and mood disorders, as well as non-neurological comorbidities, including blood hyperserotonemia, immune dysregulation, and GI dysfunction (e.g., chronic constipation, diarrhea, abdominal pain, and gastroesophageal reflux).

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans. As used herein, the term “subject” and “patient” are used interchangeably herein and refer to human.

As used herein “amount” or “level” or “abundance” refers to the amount of a particular analyte (e.g., a metabolite) present in the sample. The amount may be a number, ratio, proportion, or a percentage of the analyte compared to the control sample or determined using a standard curve. The amount may be an absolute amount or a relative amount.

As used herein, a biological sample may be of any biological tissue, fluid, or cell from the subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. Methods of collecting a biological sample from a subject are well known in the art. In some aspects, the biological sample is whole blood, plasma, platelet, RBC, or urine.

A sample from the subject can be procured one or more times and testing performed one or more times. In some aspects, samples can be procured from the subject before, during, and/or after a treatment for ASD. In some aspects, a sample can be procured from the subject prior to the start of ASD. In some aspects, sample can be procured from the subject undergoing ASD treatment. Additionally, samples can be procured repeatedly at multiple stages after initial sample procurement, to determine and/or monitor ASD in a subject.

In some aspects, a control sample can be procured from a healthy subject. In some aspects, the control can be an average of the combination of disclosed biomarker levels from different healthy sources (e.g., more than one healthy control subject and/or more than one subject has a low risk of developing irAE). In some aspects, the control sample can be a pooled sample. In some aspects, the control sample is procured from a typically developing subject or a neurotypical subject.

As used herein, a “neurotypical subject” refers to a subject determined as not having cognitive and/or behavioral disorders including ASD, ADHD, depression, and anxiety. In some aspects, a neurotypical subject does not have first-degree relatives with ASD.

### II. Method of Evaluation of Metabolites

In some aspects, the metabolites used for evaluation as described herein can be measured in a biological sample obtained from the subject. The sample may be selected from blood, plasma, red blood cells (RBC) and urine. Multiple different samples may be obtained from a subject and analyzed. The methods used for preparing and measuring the metabolites in various samples are described in further detail in: Adams et al., Nutrition & Metabolism 2011, 8:34, the disclosure of which is incorporated herein by reference in its entirety.

The subject can be, without limitation, a human, a non-human primate, a mouse, a rat, a guinea pig, and a dog. In some aspects, the subject is a human subject. The subject can be a premature newborn, a term newborn, a neonate, an infant, a toddler, a young child, a child, an adolescent, a pediatric patient, a geriatric patient. In one aspect, the subject is a child patient below about 18, 15, 12, 10, 8, 6, 4, 3, 2, or 1 year old. In another aspect, the subject is an adult patient. In another aspect, the subject is an elderly patient. In another aspect, the subject is about between 1 and 2, between 1 and 3, between 1 and 4, between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old.

Though, methods for obtaining and extracting the metabolites from a wide range of biological samples, are known in the art, the variable stability of metabolites, the source of a sample, and minor changes in procedure can have a major impact on the observed metabolites.

In some aspects, biological samples can be obtained at certain times of day and/or fast condition. For example, blood samples may be collected after an overnight fast (8-12 hours). Urine samples can be procured as for example as first morning (overnight) urine. In some aspects, a blood sample may comprise or consist of plasma, red blood cells (RBC), platelets, whole blood. In some aspects, the same metabolite(s) can be measured in one or more of samples comprising or consisting of plasma, RBC, platelets, whole blood, and urine.

In some aspects, the metabolites measured in a plasma sample from a subject is sulfate (free), nitrotyrosine, sulfate (total), uridine, glutathione, ATP, beta-amino isobutyrate, GSSG/GSH ratio, tryptophan, Carnitine (free), glutathione (oxidized), GABU, carnitine (total), biotin, glutamate, carotenes (total), SAM/SAH, taurine, vitamin C, magnesium, serine, adenosine, vitamin B5, homocysteine, homocysteine, or any combination thereof.

In some aspects, the metabolites measured in RBC sample from a subject are NADH, NADP, SAM, choline (total), iron, phosphorus, potassium, copper, calcium, cadmium, or any combination thereof.

In some aspects, the metabolites measured in platelet sample from a subject are acetylcholine, norepinephrine, serotonin, epinephrine, or any combination thereof.

In some aspects, the metabolites measured in urine sample from a subject are thallium, tin, FIGLU, antimony, lead, or any combination thereof.

In some aspects, the metabolites measured in whole blood (WB) sample from a subject are cadmium, lithium, copper, or any combination thereof.

In one aspect, the method comprises measuring the level of one, or a combination of two or more metabolites in the sample. For instance, the level of one or the levels of 2, 3, 4, 5, 6, 7, 8, 9, 10, 15, 20, 25, 30, 40, or more metabolites can be measured. The metabolites and combinations of metabolites are selected from the metabolites listed in Tables 1-6.

In some aspects, minerals and plasma amino acids can be measured using Doctor's Data (St. Charles, IL, USA—http://www.doctorsdata.com). In some aspects, vitamins can be measured using Vitamin Diagnostics (South Amboy, NJ, USA; www.europeanlaboratory.nl).

In some aspects, vitamins can be measured in the blood compartment (serum, plasma, or RBC) where they are most highly concentrated. In some aspects, if the vitamin is evenly distributed intra- and extracellularly then whole blood can be used for measurement. For e.g., fat-soluble vitamins (A, D, E, K) can be measured in serum, while water-soluble vitamins. can be measured in the plasma (e.g., vitamin C), whereas other vitamins (e.g., pantothenic acid) can be measured in whole blood. In some aspects, an approach which provides the best estimation of total body levels, can be employed. In some aspects, vitamin-specific microbiological organisms can be used for whole blood measurement with a high degree of reliability.

In some aspects, essential minerals can be measured in RBC, serum, whole blood, and urine. In some aspects, serum samples gives measurement of an average of the last several days, RBC gives measurement of an average of the last several months, and whole blood gives measurement of an average of both. In some aspects, serum minerals can be analyzed on an automated clinical chemistry analyzer (Olympus AU680, Olympus America Inc.; Centerville, Pa., USA) using for e.g., commercial assays. In some aspects, essential minerals can be measured in RBC, whole blood and/or serum depending upon which compartment is known to have the higher concentration for that mineral. For e.g., Lithium can be measured in whole blood.

In some aspects, whole blood and packed red blood cells are collected in a potassium EDTA trace metal free (royal blue top; BD Vacutainer, Franklin Lakes, NJ). Packed red blood cells is spun for 15 minutes in a centrifuge at 1500 g (g-force), the plasma and buffy coat are removed and the remaining packed red blood cells can be submitted for testing. In some aspects, elemental analysis is performed after digesting an aliquot of sample using a temperature controlled microwave digestion system (Mars5; CEM Corp; Matthews, SC). The digested sample is then analyzed by, for e.g., Mass Spectrometry. In some aspects, results can be verified for precision and accuracy using controls for e.g., controls from Doctor's Data and Seronorm whole blood controls (Sero; Billingstad, Norway).

In some aspects, mass spectrometry (MS) can be used to measure other metabolites in a sample, for e.g., plasma, urine, RBC, and whole blood. Non-limiting examples of MS that can be used to measure metabolites include Liquid chromatography-mass spectrometry (LC-MS), LCT-MS, Inductively Coupled Plasma-Mass Spectrometry (ICP-MS), and liquid chromatography-tandem mass spectroscopy (LCT-MS). In some aspects, amino acids can be measured in blood samples, using MS. Briefly, overnight fast blood samples are collected into purple top (EDTA) tubes. Blood is centrifuged within 30 minutes, and plasma is mixed with 5-sulfosalicylic acid to precipitate proteins prior to freezing. In some aspects, plasma amino acids is analyzed by a reversed phase high performance liquid chromatography (HPLC) tandem mass spectrometry (MS/MS) technique (Prostar 420 HPLC autosampler, Prostar 210 solvent delivery module, 1200 L mass spectrophotometer, Varian, Inc.; Palo Alto, CA). In some aspects, results are verified for precision and accuracy using in-house controls and a Native (Physiological) Sample Standard (Pickering Laboratories). Non-limiting examples of metabolites that can be measured using MS include nitrotyrosine, SAM, tryptophan, thallium, beta-amino isobutyrate, glutamate, cadmium, iron, phosphorus, lithium, SAM/SAH, potassium, tin, taurine, copper, magnesium, antimony, lead, serine, adenosine, calcium, cadmium, homocysteine, and homocysteine.

In some aspects, provided in the disclosure is a method of measuring metabolites using spectrophotometry. In some aspects, plasma samples procured from a subject can be evaluated using spectrophotometry. Whole blood can be collected using a blood collection tube, for e.g., BD Vacutainer® green top blood collection tube, 4 mL, NaHeparin 75 USP units. The blood samples can be centrifuged at 2000 g for 15 min, and RBC-free, platelet-depleted plasma can be collected for analysis. In some aspects, the plasma can be divided into aliquots and analyzed immediately or stored at −80° C. for later analysis. A standard curve for each metabolite can be prepared using standard solutions prepared in a buffer for e.g., phosphate buffer, PBS pH=7.0. In some aspects, buffers are treated with chelating resin to remove impurities for e.g., metals. Absorbance measurements can be made using various concentrations of the standard solutions, and the standard curve can be prepared using a median absorbance measurement obtained for each standard solution. The samples can be analyzed using a spectrophotometer using an appropriate absorbance measurement for each metabolite to be analyzed, and comparing the measurement to the standard curve to obtain the amount of metabolite in the sample. Non-limiting examples of metabolites that can be measured using spectrophotometry include free sulfate, total sulfate, glutathione, NADH, NADP, ATP, carotenes, vitamin C, epinephrine, carnitine, serotonin, gssg/gsh, uridine, norepinephrine, acetylcholine, choline, and FIGLU.

In some aspects, evaluation of the metabolites can be conducted using microbiological assays. Microbiological assays involve measuring specific growth response to presence of metabolite of a microorganism which estimate the amount of metabolite in a sample. Briefly, microbiological assays determine metabolite levels in an extract of a sample (for e.g., plasma) obtained from the subject, by measuring the growth of the metabolite sensitive microorganism in a metabolite free medium to which a known amount of the sample extract had been added. Since the growth of the sensitive microorganism is, proportional to the metabolite concentration in the medium, measuring a growth parameter, for e.g., optical density after incubation for a selected period of time and at a certain selected temperature, and comparing this parameter to values observed by running the same test with samples containing different, known concentrations of metabolite determine the concentration of metabolite in the sample extract. In some aspects, measuring the metabolite involves titratable acidity produced by microorganism as a function of the quantity of metabolite present in the sample. In some aspects, a predetermined standard curve can used to evaluate the amount of metabolite in a sample. Non-limiting examples of metabolite sensitive microorganism include, Lactobacillus casei (sensitive to biotin and folic acid), Saccharomyces cerevisiae (sensitive to biotin), Lactobacillus arabinosus (sensitive to biotin), Tetrahymena thermophila (sensitive to lipoate), Torulopsis pintolopessi (sensitive to choline), carnitine-specific mutant of the enteric yeast Torulopsis bovina (sensitive to carnitine) and Ochromonas Danica (sensitive to biotin). Non-limiting examples of metabolites that can be measured using microbiological assay include biotin, choline, vitamin B5, and carnitine.

In some aspects, metabolites can be measured using colorimetric or fluorometric assays. In some aspects, the metabolites measured using colorimetric or fluorometric assays comprise glutathione and ATP.

In some aspects, a significant difference in the level of one or combination of metabolite can be an increase or a decrease in the level of the metabolite in the sample when compared to the level of the metabolite in the control panel of metabolite levels. A significantly different level of the one or combination of metabolites can be determined by applying each of the measured levels of the metabolites against a database of metabolite control measured levels created by measuring metabolite levels of the one or more metabolite in control subjects. The database can be stored on a computer system.

In some aspects, measured metabolites have an elevated level compared to control sample. In such aspects, the metabolite have an elevated level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to a control sample. In some aspects, the metabolite having elevated level compared to control sample is nitrotyrosine, uridine, GSSG/GSH ratio, choline (total), thallium, carnitine (free), glutathione (oxidized), carnitine (total), beta-amino isobutyrate, glutamate, iron, phosphorus, potassium, tin, vitamin C, copper (whole blood and/or RBC), FIGLU, antimony, lead, serine, adenosine, homocysteine, homocystine, or any combination thereof.

In some aspects, measured metabolites have decreased level compared to control sample. In such aspects, the metabolite have a decreased level of at least about 0.01%, at least about 0.05%, at least about 0.1%, at least about 0.2%, at least about 0.3%, at least about 0.4%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100% greater as compared to a control sample. In some aspects, the metabolite having decreased level compared to control sample is sulfate (free), sulfate (total), glutathione, NADH, acetylcholine, NADP, ATP, SAM, norepinephrine, serotonin, tryptophan, GABU, biotin, epinephrine, carotenes (total), cadmium, lithium, SAM/SAH, taurine, magnesium, calcium, vitamin B5, cadmium, or any combination thereof.

### III. Method of Diagnosis and Treatment

One aspect of the present disclosure encompasses a method of diagnosing Autism Spectrum Disorder (ASD) in a subject suspected of having or at risk of having ASD. The method comprises the steps of (a) measuring levels of a combination of two or more metabolites selected from the metabolites listed in Table 1 in a biological sample obtained from the subject; (b) applying the measured levels of each metabolite against a control panel of metabolite levels created by measuring metabolite levels of the one or combination of metabolites in control typically developing (TD) subjects; and indicating an ASD diagnosis if the levels of each of the two or more metabolites in the biological sample are significantly different from the levels of the two or more metabolites in the control panel of metabolite levels if the AUROC value for the combination of metabolites is about 0.85 or higher. The applying comprises calculating the sensitivity (TPR) and specificity (TNR) for the combination of metabolites using Fisher Discriminant Analysis (FDA) or support vector machines (SVM); and calculating the area under the curve (AUC) of the receiver operating characteristic (ROC) curve (AUROC) for the combination of two or more metabolites using the FPR and TPR calculated in step (b). The biological sample can be whole blood, plasma, red blood cells (RBCs), urine, or any combination thereof. The metabolite can be a metabolite associated with the one or combination of two or more metabolites identified as having a level in the biological sample significantly different from the level of the one or combination of metabolites in the control sample.

The metabolites listed in Table 1 can be identified by comparing the measured level of the metabolite in the biological sample to the level of the metabolite in the control panel of metabolite levels using a univariate statistical analysis method selected from hypothesis testing, evaluating the area under the receiver operator curve (AUROC) values, or a combination of both.

Hypotheses testing can be performed by evaluating the type of distribution for each of the ASD and TD groups' measurements and selecting an appropriate parametric or non-parametric test. A parametric or non-parametric test can be selected by determining the normality and variance of each individual clinical measurement variable for both the ASD and TD groups separately. Further, a parametric test can be performed if a normality assumption holds true for the ASD and TD groups. An equal variance t-test or Welch's test (unequal variance t-test) can be performed if the observed variance is significantly different between the ASD and TD groups. In some aspects, a Mann-Whitney test is performed if the ASD and TD groups follow the same non-parametric distribution. In some aspects, the ASD and TD groups are adjusted by their means and subjected to the Kolmogorov-Smirnov test where different distributions are observed. In some aspects, the method further comprises determining the false discovery rate (FDR) for each measurement using a leave-one-out (L-1-O) approach.

The combination of two or more metabolites can be identified by performing multivariate analysis using Fisher Discriminant Analysis (FDA) and support vector machines (SVM) and selecting the combinations of two or more metabolites comprising the best FDA or SVM measure for each combination of elements.

Performing FDA can comprise the steps of (a) evaluating all possible combinations of two, three, and four-metabolites from among the metabolites of Table 1; (b) examining the fitted AUROC and performance when subjected to cross-validation using leave-one-out cross-validation; and (c) calculating the area under the AUROC value for each combination of two, three, and four-metabolites. The combination of two or more metabolites can comprise the combinations of two, three, and four-metabolites comprising the highest 1000 AUROC values following leave-one-out cross-validation. The method can further comprise using a greedy algorithm to identify combinations of metabolites comprising five or more metabolites. In some aspects, performing SVM comprises assessing all possible combinations of 5-metabolites and subjecting each combination of five or more metabolites leave-one-out cross-validation if the combination of five or more metabolites attains an accuracy greater than 0.90.

The combination of two or more metabolites can comprise a combination of metabolites of Table 7 and Table 2. In some aspects, the control panel of metabolite levels is stored on a computer system. The method can diagnose ASD at birth or pre-birth.

In some aspects, the levels of a combination of two metabolites are measured. The combination of two metabolites can be selected from the combinations of metabolites listed in Table 7.

In some aspects, the levels of a combination of three metabolites are measured. In one aspect, the levels of the three metabolites are the levels of free sulfate in plasma, the level of uridine in plasma, and the level of beta-amino isobutyrate in plasma.

In some aspects, the levels of a combination of four metabolites are measured. In one aspect, the levels of the four metabolites are the level of free sulfate in plasma, the level of uridine in plasma, the level of homo cystine in plasma, and the level of beta-amino isobutyrate in plasma.

In some aspects, the levels of a combination of five metabolites are measured. In one aspect, the combinations of five metabolites are the levels of the combinations of five metabolites listed in Table 2. In one aspect, the levels of the five metabolites are the level of free sulfate in plasma, the level of uridine in plasma, the level of initial homo cystine in plasma, the level of beta-amino isobutyrate in plasma, and the level of magnesium in the serum. In other aspects, the levels of the five metabolites are the level of free sulfate in plasma, the level of uridine in plasma, the level of homo cystine in plasma, the level of beta-amino isobutyrate in plasma, and the level of tryptophan in the plasma. In yet other aspects, the levels of the five metabolites are the level of free sulfate in plasma, the level of magnesium in the serum, the level of homo cystine in plasma, the level of uridine in plasma, and the level of beta-amino isobutyrate in plasma.

In some aspects, the levels of a combination of six metabolites is measured. In one aspect, the levels of the six metabolites are the level of free sulfate in plasma, the level of homo cystine in plasma, the level of uridine in plasma, the level of beta-amino isobutyrate in plasma, the level of magnesium in the serum, and the level of copper in RBCs.

In some aspects, the method can diagnose ASD with high level of sensitivity and specificity. In some aspects, the method can diagnose ASD in a subject with a sensitivity greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or even with a 100% sensitivity. In some aspects, the method can diagnose ASD in a subject with a specificity greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or even with a 100% specificity. In some aspects, the method can diagnose ASD with a specificity of at least about 80% to 90%, a specificity of at least about 80% to 90%, or both. In one aspect, the method can diagnose ASD with a specificity and sensitivity of 100%.

In some aspects, methods disclosed herein can diagnose ASD with a low misclassification error, such as a misclassification error of about 10, 8, 9, 7, 6, 5, 4, 3, 2, or about 1% or even with no misclassification error. In some aspects, the method can diagnose ASD with a misclassification error of about 5% or less, or about 3% or less. In further aspects, the method can diagnose ASD with an accuracy of about 75, 80, 85, 90, 95% or with about 100% accuracy. In some aspects, the method can diagnose ASD with an accuracy of about 95% or higher, such as with an accuracy of about 97% or with about 100% accuracy.

The method can further comprise assigning a medical, behavioral, and/or nutritional treatment protocol to the subject based on metabolites in the biological sample that are significantly different from the level of the metabolite in the control panel of metabolite levels if the AUROC value for the combination of metabolites is about 0.85 or higher. In some aspects, the treatment protocol can comprise adjusting the level of one or a combination of two or more metabolites in the subject. The treatment protocol can comprise administering a combination of anti-oxidants and a source of sulfate. In some aspects, treatment protocol can comprise administering risperidone (sold under Risperdal®) and/or aripiprazole (sold under Ability®). In some aspects, the treatment protocol can comprise, behavioral (for e.g., Applied Behavior Analysis (ABA), Discrete Trial Training (DTT), Pivotal Response Training (PRT)), developmental for e.g., speech and language therapy, occupational therapy, sensory integration therapy, physical therapy, early start Denver model (ESDM)), educational (for e.g., Treatment and Education of Autistic and Related Communication-Handicapped Children (TEACCH)), social-relational (for e.g., Relationship Development Intervention), psychological (for e.g., Cognitive-Behavior Therapy (CBT)), alternative treatment, or any combination thereof.

In some aspects, the treatment protocols can comprise restoring the level of one or more metabolite identified as significantly different in the biological sample obtained from the subject to a level of the one or more metabolites in the control panel of metabolite levels obtained from TD individuals with no GI problems. Similarly, when a metabolite represents a group of metabolites correlated with the metabolite, the treatment protocol can comprise restoring the level of one or more of the group of metabolites associated with the identified metabolite. The metabolite can be supplemented by nutritional means, or by oral or parenteral administration of compositions comprising the metabolite. The level of a metabolite can be restored by about 20%, 30%, 40%, 50%, 60%, 70%, 80%, 90% or more.

Another aspect of the instant disclosure encompasses a method of determining a personalized treatment protocol for a subject suspected of having or at risk of having ASD. The method comprises diagnosing ASD in a subject suspected of having or at risk of having ASD, and assigning a personalized medical, behavioral, or nutritional treatment protocol to the subject if the subject is diagnosed with ASD. Diagnosing ASD can be as described above. The treatment protocol can comprise administering a combination of anti-oxidants and a source of sulfate. In some aspects, treatment protocol can comprise administering therapeutic compound risperidone (sold under Risperdal®) and/or aripiprazole (sold under Ability®). In some aspects, the treatment protocol can comprise, behavioral (for e.g., Applied Behavior Analysis (ABA), Discrete Trial Training (DTT), Pivotal Response Training (PRT)), developmental for e.g., speech and language therapy, occupational therapy, sensory integration therapy, physical therapy, early start Denver model (ESDM)), educational (for e.g., Treatment and Education of Autistic and Related Communication-Handicapped Children (TEACCH)), social-relational (for e.g., Relationship Development Intervention), psychological (for e.g., Cognitive-Behavior Therapy (CBT)), alternative treatment, or any combination thereof.

In some aspects, a treatment protocol can be personalized based on the metabolites found to be significantly different in a sample obtained from the subject when compared to a control and identified using the method described herein. Such a personalized treatment protocol can include adjusting in the subject the level of the one or combination of metabolites found to be identified as being significantly different in the biological sample obtained from the subject to a level of the one or more metabolites in the control panel of metabolite levels obtained from TD individuals. The treatment protocol can also include adjusting the levels of one or more metabolite associated with the one or combination of two or more metabolites identified as having a level in the biological sample significantly different from the level of the one or combination of metabolites in the control sample.

Yet another aspect of the instant disclosure encompasses a method of monitoring a therapeutic effect of an ASD treatment protocol in a subject suspected of having or at risk of having ASD. The method comprises the steps of (a) measuring in a first biological sample obtained from the subject the level of one or a combination of two or more metabolites selected from the metabolites listed in Table 1 and any combination thereof; (b) measuring in a second biological sample obtained from the subject at a period of time after the first biological sample is obtained the level of the one or combination of two or more metabolites; and (c) comparing the level of the one or combination of two or more metabolites in the first sample and the second sample. Maintenance of the level of the one or combination of two or more metabolites or a change of the level of the one or combination of two or more metabolites to a level of the one or combination of two or more metabolites in a control panel of metabolite levels created by measuring metabolite levels of the one or combination of two or more metabolites in control TD subjects is indicative that the treatment protocol is therapeutically effective in the subject.

In some aspects, the method monitoring a therapeutic effect of an ASD treatment protocol comprises modifying the treatment protocol. For example, in an aspect, a treatment protocol can be altered by changing the amount of one or more of the therapeutic compounds, or by changing the treatment protocol, or changing the frequency of administration of therapeutic compounds, or by changing the duration of time one or more of the therapeutic protocol administered to a subject.

Another aspect of the instant disclosure encompasses a method of assessing the behavioral severity of ASD in a subject. The method comprises the steps of (a) measuring levels of a combination of two or more metabolites selected from the metabolites listed in Table 1 in a biological sample obtained from the subject; (b) applying the measured levels of each metabolite against a control panel of metabolite levels created by measuring metabolite levels of the one or combination of metabolites in control typically developing (TD) subjects; and indicating the subject as having high behavioral severity, or at risk for having behavioral severity if the levels of each of the two or more metabolites in the biological sample are significantly different from the levels of the two or more metabolites in the control panel of metabolite levels if the AUROC value for the combination of metabolites is about 0.85 or higher. The applying comprises calculating the sensitivity (TPR) and specificity (TNR) for the combination of metabolites using Fisher Discriminant Analysis (FDA) or support vector machines (SVM); and calculating the area under the curve (AUC) of the receiver operating characteristic (ROC) curve (AUROC) for the combination of two or more metabolites using the FPR, TPR and TNR calculated in step (b). The biological sample can be whole blood, plasma, red blood cells (RBCs), urine, or any combination thereof. The metabolite can be a metabolite associated with the one or combination of two or more metabolites identified as having a level in the biological sample significantly different from the level of the one or combination of metabolites in the control sample.

In some aspects, the method of assessing the behavioral severity of ASD in a subject comprise measuring levels of a combination of free sulfate in plasma and iron in red blood cells. In some aspects, the method of assessing the behavioral severity of ASD in a subject comprise measuring levels of a combination of at least free sulfate in plasma and iron in red blood cells.

In further aspects, the method of assessing the behavioral severity of ASD in a subject, comprise determining a personalized treatment protocol for the subject having behavioral severity, or at risk of having behavioral severity. The method comprises assigning a personalized medical, behavioral, or nutritional treatment protocol to the subject. Diagnosing ASD can be as described above. In some aspects, high behavioral severity comprise inability to use spoken language, extreme sensitivity to crowds, bright lights, or loud noise, lower IQ, repetitive behaviors, and physical symptoms like sleeplessness and epilepsy. The treatment protocol can comprise administering a combination of anti-oxidants and a source of sulfate. In some aspects, treatment protocol can comprise administering therapeutic compound risperidone (sold under Risperdal®) and/or aripiprazole (sold under Ability®). In some aspects, the treatment protocol can comprise, behavioral (for e.g., Applied Behavior Analysis (ABA), Discrete Trial Training (DTT), Pivotal Response Training (PRT)), developmental for e.g., speech and language therapy, occupational therapy, sensory integration therapy, physical therapy, early start Denver model (ESDM)), educational (for e.g., Treatment and Education of Autistic and Related Communication-Handicapped Children (TEACCH)), social-relational (for e.g., Relationship Development Intervention), psychological (for e.g., Cognitive-Behavior Therapy (CBT)), alternative treatment, or any combination thereof.

### IV. Kit

An additional aspect of the instant disclosure encompasses a kit for diagnosing Autism Spectrum Disorder (ASD) in a subject suspected of having or at risk of having ASD, determining a personalized treatment protocol, monitoring a therapeutic effect of an ASD treatment protocol, or any combination thereof. The kit comprises: (a) a container for collecting a biological sample from the subject; (b) solutions and solvents for preparing an extract from a biological sample obtained from the subject; and (c) instructions for (i) preparing the extract, (ii) measuring the level of one or more metabolites selected from the metabolites listed in Table 1; and (iii) applying the measured metabolite levels against a control panel of metabolite levels obtained from typically developing (TD) individuals.

In some aspects, the disclosed kit comprise a collection of elements including at least one non-standard laboratory reagent for use in the disclosed methods, in appropriate packaging, optionally containing instructions for use. A kit may further include any other components required to practice the methods, such as dry powders, concentrated solutions, or ready-to-use solutions. In some aspects, a kit comprises one or more containers that contain reagents for use in the methods. Containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

In some aspects, a kit further comprises instructions for testing a biological sample of a subject having or at risk of having ASD. The instructions will generally include information about the use of the kit in the disclosed methods. In other aspects, the instructions may include at least one of the following: description of possible therapies including therapeutic agents; clinical studies; and/or references. The instructions may be printed directly on the container (when present), or as a label applied to the container, or as a separate sheet, pamphlet, card, or folder supplied in or with the container.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Study Design and Methods

This experiment was designed to re-examine blood and urine measurements collected from a study with two cohorts of children (ASD and TD) using a multivariate statistical approach with the ultimate goal to better understand pathophysiology underlying ASD. Thus, there are several primary objectives of this work. One key focus is to expand the understanding of the relationship between metabolic factors and the capability to predict ASD. By comprehensively evaluating multiple measurement panels for their capacity to accurately predict diagnosis, the efficacy of candidate biomarkers can be holistically examined. Furthermore, the identification of relationships between metabolites and behavioral symptoms allows for the identification of mechanisms of interest pertinent to better understanding ASD etiology.

In total, the dataset comprised 155 different measurement quantities collected as part of a nutritional and metabolic study involving 99 individuals, which was conducted with the approval of the Human Subjects Institutional Review Board of Arizona State University. Amino acids, essential nutrients and vitamins were reported in the study. Of the 99 study participants, 55 had an ASD diagnosis while 44 were developing typically. The participants' ages ranged from 5-16 years, with an average age of 10.4 years. The proportion of males (89%) to females (11%) was the same for both ASD and TD cohorts. Participants were selected such that none had received vitamin/mineral supplements in the last 2 months. Given the focus of this work, only the baseline data, before any interventions were started, are used here.

One inclusion criterion for the ASD group was that all participants had to have been previously diagnosed with ASD by a psychiatrist or comparable clinical professional. The participants in the typically developing group were in good mental and physical health and to have no evidence suggesting Attention Deficit Disorder, based on parent characterization. Initial ASD symptom severity was measured via the Pervasive Development Disorder Behavior Inventory (PDD-BI) modified Autism Composite, Severity of Autism Scale (SAS) and Autism Treatment Evaluation Checklist (ATEC).

Levels of several neurotransmitters in platelets, and levels of carnitine and acetyl-carnitine in plasma were reported. Morning blood and urine samples were collected after an overnight fast for all children. Doctor's Data was used for performing the analysis of minerals and plasma amino acids via liquid chromatography-tandem mass spectroscopy (LCT-MS). Vitamins and other biomarkers were analyzed by Vitamin Diagnostics using spectrophotometry and microbiological assays. Essential minerals were measured in RBC, serum, and whole blood, while amino acids were measured in plasma.

### Analysis Methodology

Univariate Analysis. Initial univariate analysis was performed using both hypothesis testing and evaluating the area under the receiver operator curve (AUROC) values. The receiver operator curve (ROC) is produced by plotting the False Positive Rate (FPR) vs the True Positive Rate (TPR) when determining thresholds to classify between two groups. As the integral of the ROC, the AUROC provides a measure of how well the characteristic or variable in question classifies between two different groups. For the purposes of this analysis, the measurements observed for each metabolite, element or xenobiotic compound were treated as scores to classify between the ASD and TD cohorts. Subsequently, all possible variables were examined individually for their capability to have set thresholds to separate between the two groups. Individuals with missing data were omitted from the analysis.

Hypothesis testing was performed by evaluating the type of distribution for each of the cohorts' measurements and then selecting the appropriate parametric or non-parametric test. The normality and variance of each individual clinical measurement variable were determined for both the ASD and TD group separately. When the normality assumption was determined to hold true for both groups, a parametric test was performed. Either an equal variance t-test or Welch's test (unequal variance t-test) was performed depending on if the variance observed was significantly different between the groups.

A Mann-Whitney test was used if the two groups were observed to follow the same non-parametric distribution. In cases where different distributions were observed, both groups were adjusted by their means and then subjected to the Kolmogorov-Smirnov test. If the same distribution was observed in both groups, the Mann-Whitney test was applied, otherwise the Welch's test was used.

To account for the multiple testing problem, the false discovery rate (FDR) for each of the measurements was determined. FDR is defined as the expected proportion of discoveries that can be defined as being falsely rejected. To determine the FDR for each significant clinical measurement variable, the leave-one-out (L-1-O) approach was used.

Correlation Analysis. Metabolites, elements, and xenobiotics that had been determined to be significant via univariate testing were further examined using correlation analysis. The Pearson correlation coefficients between all significant variables were determined with pairs attaining a p-value value less than 0.05 subject to L-1-O FDR. Those relationship pairs that were able to achieve an FDR less than 0.10 were deemed to be significant. The correlations between all identified metabolites were determined for both the ASD and TD groups separately as well as combined. Behavioral symptoms associated with ASD as measured by SAS and PDI-R were examined in the context of their relationship to significant metabolite measurements taken.

Multivariate Analysis Preprocessing. In order to perform a thorough multivariate analysis, imputation had to be performed so that it is possible to include even individuals lacking measurements for some fields. Common single imputation techniques such as hot deck and mean substitution will attenuate having an accurate impression of the population a dataset is sampled from and will reduce the significance of any of the correlations between variables measured to each other. To account for this problem, a multiple imputation approach was used in conjunction with the multivariate Fisher Discriminant Analysis (FDA) and support vector machines (SVM).

The use of multiple imputation techniques consists of three main steps. Samples were repeatedly drawn from a known distribution, subjected to statistical analysis and subsequently all findings are pooled across runs. A probability density function was estimated from existing data for both the ASD and TD groups. Values were then selected from this distribution and used to impute the missing measurements. Subsequently, FDA and SVM were performed using the complete dataset with the imputed values included. FDA was repeated 100 times for each model that met certain AUROC fit threshold criteria, and the results for classification as evaluated by AUROC were averaged. An optimized 5-variable FDA model was also determined using only those variables that had no instances of missing data.

Multivariate Analysis. FDA was used to develop models based on multiple variables for differentiating the ASD and TD groups. FDA is defined as a dimensionality reduction technique that seeks to separate classes by finding a projection where such differences are maximized, while differences in the same group are minimized. The objective function for FDA is:

$$J ( W ) = \frac{{W}^{T} {S}_{B} W}{{W}^{T} {S}_{W} W}$$

where the between class scatter (SB) is maximized and within class scatter (Sw) is minimized.

FDA Application. All possible 2, 3 and 4-biomarker panels were evaluated from among the 46 biochemical and xenobiotic compounds that had been shown to be statistically significant via univariate testing. For each run, the fitted AUROC and performance when subjected to cross-validation was examined. Those with the highest 1000 AUROC values following leave-one-out cross-validation were retained for use in a greedy algorithm approach towards uncovering variable panels with more constituents. The greedy algorithm is used for combinations above 4 variables, to reduce computational cost. The top 1000 4-variable models served as the basis for 5-biomarker panels by adding back variables from those 42 that were statistically significant yet not previously selected. This approach was repeated to develop models containing 6 biomarkers as well. Additionally, statistics regarding the top 1000 5-variable models and 6-variable models were also noted.

SVM Analysis. Support Vector Machines (SVM) is a machine-learning technique which was also used to develop models to differentiate ASD and TD groups. Measurement variables that had been deemed to be statistically significant were examined using an exhaustive classification approach. All possible combinations of 5-variables were assessed and subject to leave-one-out cross-validation if they could attain an accuracy greater than 0.90. The variables that appeared frequently in panels that passed this benchmark were recorded.

### Example 1. Univariate Analysis of Metabolomic and Nutritional Profiles Reveal Significant Differences Among Children with Autism Spectrum Disorder and Typically Developing (TD) Individuals

Of the 155 initial measurements, univariate analysis revealed for the first time that 50 variables that were significantly different between the ASD and TD groups (p-value<0.05). Also for the first time, among these 50, 46 were characterized as statistically significant when also considering multiple hypothesis testing involving FDR (<0.1). From those 46 measurements that were deemed statistically significant, 7 attained AUROC values greater than 0.80, indicating moderate capability to distinguish between the ASD and TD cohorts. Specifically, free sulfate in serum, nitrotyrosine, total sulfate in serum, serum uridine, glutathione, NADH and acetylcholine were identified as meeting this criterion (Table 1). Free sulfate in serum was able to achieve the highest AUROC, with a value of 0.90.

TABLE 1
Univariate and Correlation Analysis Results Ordered by AUROC. Univariate analysis was
performed by both determining the optimal statistical test to perform to compare the
ASD and TD groups as well as calculating the AUROC between them. FDR was determined
using the leave-one-out approach to determine the robustness of each of the findings.

| | | Mean ASD | | | | | |
|---|---|---|---|---|---|---|---|
| | | value vs | | | | | |
| | | Mean TD | P- | Test- | ASD | TD | |
| Name | Source | value | value | Type | Correlations | Correlations | AUROC |
| Sulfate | Plasma | ↓ | 0 | Welch's | 12 | 18 | 0.90 |
| (Free) | | | | test***** | | | |
| Nitrotyrosine | Plasma | ↑ | 0 | Welch's | 11 | 19 | 0.87 |
| | | | | test***** | | | |
| Sulfate | Plasma | ↓ | 0 | Welch's | 12 | 19 | 0.85 |
| (Total) | | | | test***** | | | |
| Uridine | Plasma | ↑ | 0 | Welch's | 5 | 10 | 0.85 |
| | | | | test***** | | | |
| Glutathione | Plasma | ↓ | 0 | Welch's | 11 | 16 | 0.85 |
| | | | | test***** | | | |
| NADH | RBC | ↓ | 0 | Welch's | 12 | 16 | 0.84 |
| | | | | test***** | | | |
| Acetylcholine | platelets | ↓ | 0 | Welch's | 14 | 16 | 0.81 |
| | | | | test***** | | | |
| NADP | RBC | ↓ | 0 | Welch's | 11 | 19 | 0.79 |
| | | | | test [symbol] | | | |
| ATP | Plasma | ↓ | 0 | Welch's | 5 | 14 | 0.77 |
| | | | | test***** | | | |
| SAM | RBC | ↓ | 0 | t-test | 4 | 14 | 0.77 |
| Norepinephrine | platelets | ↓ | 0 | Welch's | 0 | 16 | 0.76 |
| | | | | test***** | | | |
| GSSG/GSH | Plasma | ↑ | 0 | Welch's | 10 | 18 | 0.75 |
| Ratio | | | | test***** | | | |
| Choline | RBC | ↑ | 0 | t-test | 2 | 16 | 0.75 |
| (Total) | | | | | | | |
| Serotonin | platelets | ↓ | 0 | Welch's | 0 | 13 | 0.75 |
| | | | | test***** | | | |
| Tryptophan | Plasma | ↓ | 0 | Welch's | 0 | 1 | 0.75 |
| | | | | test***** | | | |
| Thallium | Urine | ↑ | 0 | Welch's | 1 | 2 | 0.73 |
| | | | | test***** | | | |
| Free | Plasma | ↑ | 0 | t-test | 3 | 5 | 0.71 |
| Carnitine | | | | | | | |
| Oxidized | Plasma | ↑ | 0 | Welch's | 6 | 9 | 0.7 |
| glutathione | | | | test***** | | | |
| GABU | Plasma | ↓ | 0 | Welch's | 11 | 16 | 0.7 |
| | | | | test***** | | | |
| Total | Plasma | ↑ | 0 | t-test | 4 | 4 | 0.69 |
| Carnitine | | | | | | | |
| (carnitine + | | | | | | | |
| acetyl- | | | | | | | |
| carnitine) | | | | | | | |
| Beta-amino | GI | ↑ | 0 | Welch's | 0 | 3 | 0.69 |
| isobutyrate | | | | test***** | | | |
| Biotin | Plasma | ↓ | 0 | Welch's | 0 | 7 | 0.68 |
| | | | | test [symbol] | | | |
| Glutamate | Plasma | ↑ | 0.01 | Welch's | 1 | 3 | 0.68 |
| | | | | test***** | | | |
| Epinephrine | Platelets | ↓ | 0 | Mann- | 9 | 15 | 0.67 |
| | | | | Whitney | | | |
| Total | Plasma | ↓ | 0.01 | Welch's | 4 | 1 | 0.67 |
| Carotenes | | | | test***** | | | |
| Cadmium | WB | ↓ | 0 | Welch's | 5 | 1 | 0.67 |
| | | | | test***** | | | |
| Iron | RBC | ↑ | 0 | Welch's | 7 | 7 | 0.67 |
| | | | | test***** | | | |
| Phosphorus | RBC | ↑ | 0 | Welch's | 7 | 3 | 0.66 |
| | | | | test***** | | | |
| Lithium | WB | ↓ | 0.04 | Welch's | 2 | 3 | 0.66 |
| | | | | test***** | | | |
| SAM/SAH | Plasma | ↓ | 0.01 | Welch's | 6 | 15 | 0.65 |
| | | | | test***** | | | |
| Potassium | RBC | ↑ | 0.01 | Welch's | 5 | 3 | 0.65 |
| | | | | test***** | | | |
| Tin | Urine | ↑ | 0.01 | Mann- | 1 | 2 | 0.65 |
| | | | | Whitney | | | |
| Taurine | Plasma | ↓ | 0.01 | Welch's | 5 | 7 | 0.65 |
| | | | | test***** | | | |
| Vitamin C | Plasma | ↑ | 0.03 | t-test | 1 | 4 | 0.64 |
| Copper | WB | ↑ | 0.02 | t-test | 0 | 1 | 0.64 |
| FIGLU | Urine | ↑ | 0.03 | t-test | 1 | 3 | 0.63 |
| Copper | RBC | ↑ | 0.03 | t-test | 0 | 1 | 0.63 |
| Magnesium | Plasma | ↓ | 0.02 | Mann- | 0 | 3 | 0.63 |
| | | | | Whitney | | | |
| Antimony | Urine | ↑ | 0.03 | Mann- | 0 | 0 | 0.63 |
| | | | | Whitney | | | |
| Lead | Urine | ↑ | 0.02 | Mann- | 1 | 1 | 0.63 |
| | | | | Whitney | | | |
| Serine | Plasma | ↑ | 0.04 | Welch's | 0 | 3 | 0.63 |
| | | | | test***** | | | |
| Adenosine | Plasma | ↑ | 0.01 | Welch's | 4 | 10 | 0.62 |
| | | | | test***** | | | |
| Calcium | RBC | ↓ | 0.02 | Welch's | 8 | 6 | 0.61 |
| | | | | test***** | | | |
| Vitamin B5 | Plasma | ↓ | 0.02 | Welch's | 0 | 8 | 0.61 |
| | | | | test [symbol] | | | |
| Cadmium | RBC | ↓ | 0.01 | Mann- | 6 | 6 | 0.6 |
| | | | | Whitney | | | |
| Homocysteine + | Plasma | ↑ | 0.02 | Mann- | 5 | 1 | 0.6 |
| homocystine | | | | Whitney | | | |

*****Indicates case where two different non-parametric distributions were observed [symbol]
indicates data missing or illegible when filed

### Example 2. Correlation Analysis of Metabolomic and Nutritional Profiles Reveal Significant Differences Among Children with Autism Spectrum Disorder and Typically Developing (TD) Individuals

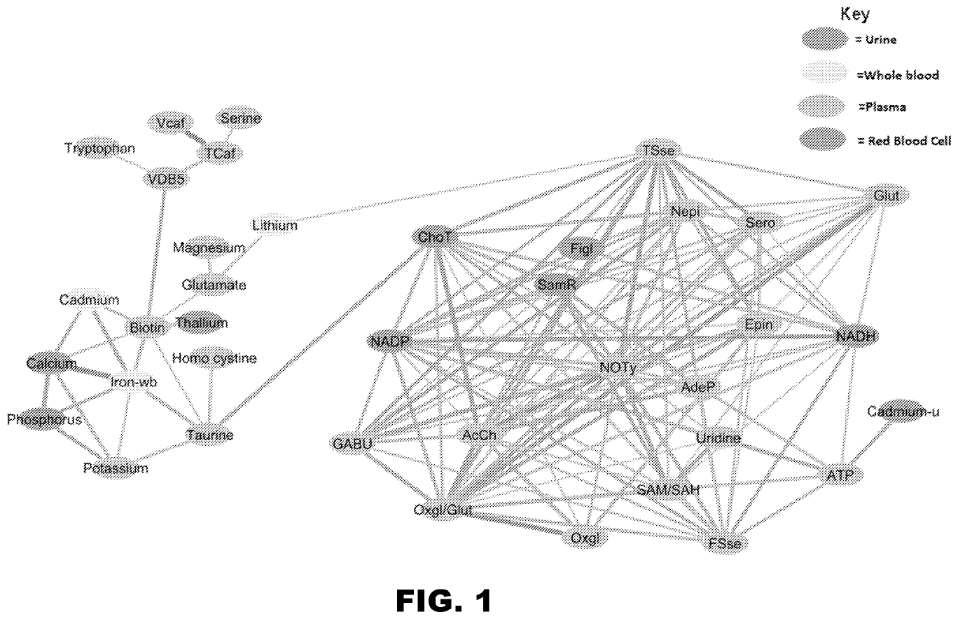

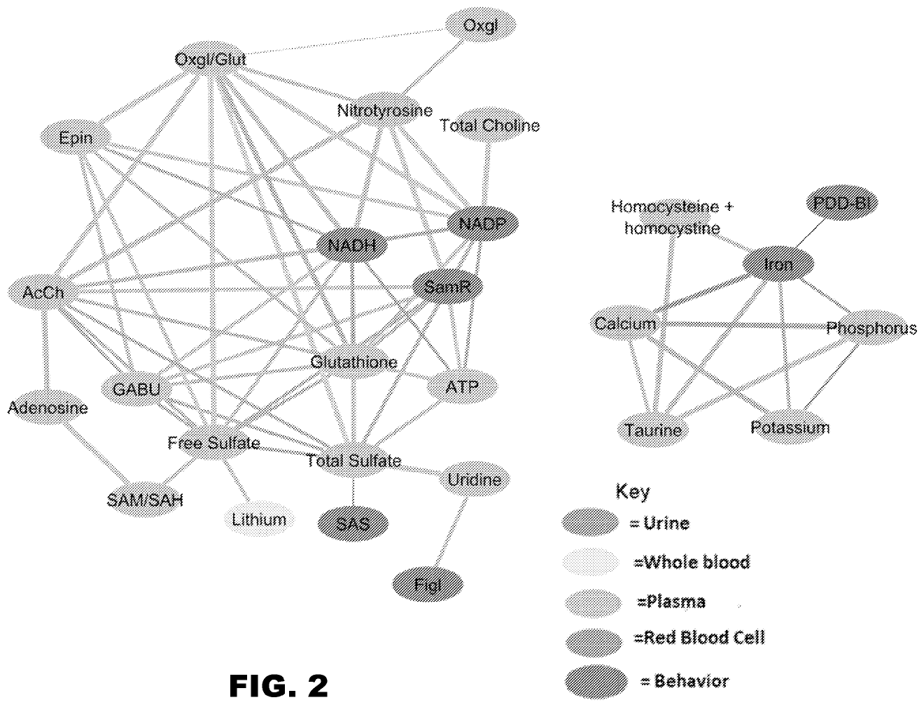

The relationship network for all significant variables was determined using correlation analysis and L-1-O FDR for each group separately. In total, there were 148 shared correlation pairs between the ASD and TD groups, when using FDR<0.10 and a Pearson correlation coefficient greater in magnitude than 0.35 (FIG. 1-2). Notable differences were observed between the ASD and TD correlation network for 294 relationships, which corresponded to 230 unique interactions in the TD cohort and 64 unique ASD interactions. The correlations between behavioral symptom severity and metabolites of significance were also included as part of this analysis. ASD severity was quantified using the SAS and PDD-BI, which were subsequently found to be significantly correlated with free sulfate in plasma and iron in red blood cells (RBC-iron), respectively (r=0.36, r=−0.38).

Generally, the TD group was observed to have a greater number of correlations across most significant metabolites and xenobiotics. However, there were exceptions to this observation for homocysteine, cadmium, phosphorus, potassium and calcium. Nonetheless, there was a considerable degree of overlap between observed relationships for the ASD cohort. About 70% of relationships present when examining the ASD cohort were also present in the TD cohort as well. The magnitudes of the relationships were also largely in concordance.

Both free sulfate in plasma and total sulfate in plasma (TSse) were among the metabolites that demonstrated the highest AUROC, indicating strong utility for separating between the ASD and TD cohorts. Further, the relationship between these metabolites to others was looked at in more detail. The correlation between each of the 44 remaining significant variables was individually assessed with regards to both free sulfate and TSse in the ASD cohort. In both the case of free sulfate and TSse, there were a greater number of significant correlations observed in the TD cohort, with most relationships overlapping. The few instances in which the ASD group was observed to have significant correlations that were not observed in the TD group for free sulfate were uridine and GABA in urine (GABU).

### Example 3. FDA Multivariate Models Reveal Combination of 2, 3, 4, and 5 Metabolites Achieved High Cross-Validated AUROC Scores

FDA multivariate models were derived using the variables that had been deemed statistically significant. Measurements for 20 of the 47 significant variables were incomplete for all individuals, which necessitated the need for multiple imputation. However, the extent of missing data was minimal, with fewer than 5 out of 99 participants missing data points for any measurement. FDA models were also derived from only the participants with complete sample sets using the same model discovery protocol as was used for the complete dataset. FDA models with 2, 3, 4, and 5 metabolites achieved very high cross-validated AUROC scores of 0.93, 0.96, 0.97, and 0.98 (Table 2). The model composition and performance were largely the same between both the full dataset and the subset of 20 variables without missing measurements (Table 2).

TABLE 2
FDA and SVM models that achieved the highest AUROC following cross-validation
(CV) for each number of potential biomarkers. CV AUROC was calculated by
using leave-one-out cross-validation and performing multiple imputation when
needed (Except in the * model, see details in Table 6). Sensitivity and specificity
are provided for the optimal operating point of the CV ROC curve.

| Number of | | | Fitted | CV | Sensitivity | Specificity |
|---|---|---|---|---|---|---|
| Markers | Method | Model Constituents | AUROC | AUROC | (TPR) | (TNR) |
| 2 | FDA | Free sulfate (plasma) | 0.92 | 0.93 | 0.94 | 0.86 |
| | | Uridine (plasma) | | | | |
| 3 | FDA | Free sulfate (plasma) | 0.95 | 0.96 | 0.92 | 0.89 |
| | | Uridine (plasma) | | | | |
| | | Beta-amino isobutyrate | | | | |
| 4 | FDA | Free sulfate (plasma) | 0.96 | 0.97 | 0.93 | 0.91 |
| | | Uridine (plasma) | | | | |
| | | Homo cystine | | | | |
| | | Beta-amino isobutyrate | | | | |
| 5 | FDA | Free sulfate (plasma) | 0.96 | 0.98 | 0.95 | 0.95 |
| | | Uridine (plasma) | | | | |
| | | Initial Homo cystine | | | | |
| | | Beta-amino isobutyrate | | | | |
| | | Serum Magnesium | | | | |
| 5*** | FDA | Free sulfate (plasma) | 0.96 | 0.97 | 0.93 | 0.89 |
| | | Uridine (plasma) | | | | |
| | | Beta-amino isobutyrate | | | | |
| | | Tryptophan (plasma) | | | | |
| | | Homo cystine (plasma) | | | | |
| 5 | SVM | Free sulfate (plasma) | 1.00 | 0.92 | 0.91 | 0.92 |
| | | Magnesium (Serum) | | | | |
| | | Homo cystine (Plasma) | | | | |
| | | Uridine (Plasma) | | | | |
| | | Beta-amino isobutyrate | | | | |
| 6 | FDA | Free sulfate (plasma) | 0.97 | 0.98 | 0.95 | 0.95 |
| | | Uridine (plasma) | | | | |
| | | Homo cystine (plasma) | | | | |
| | | Beta-amino isobutyrate | | | | |
| | | Serum Magnesium | | | | |
| | | RBC Copper | | | | |

***Represents case in which only 27 metabolites without missing data were utilized.

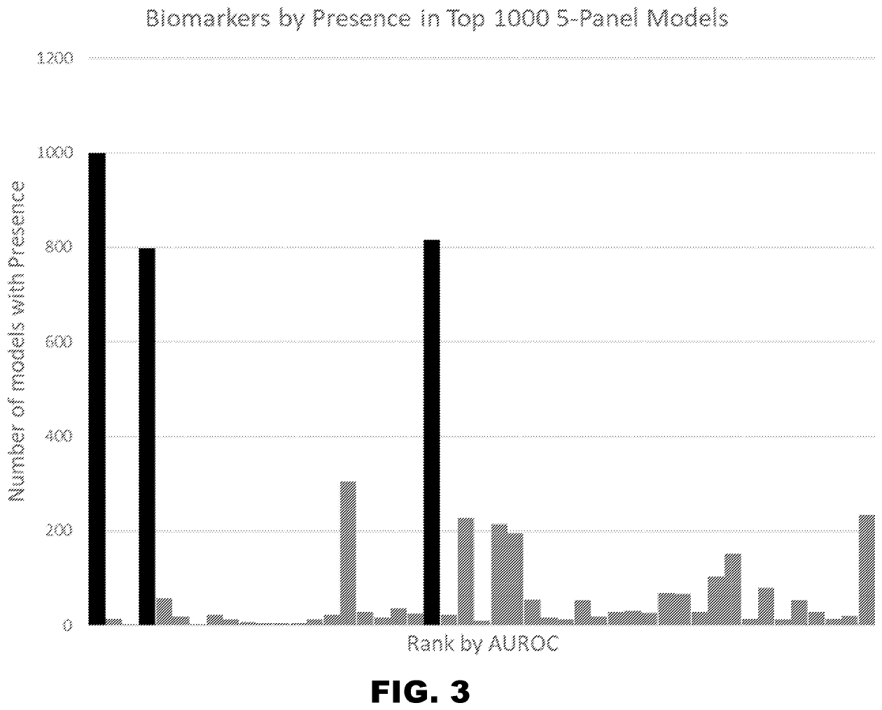

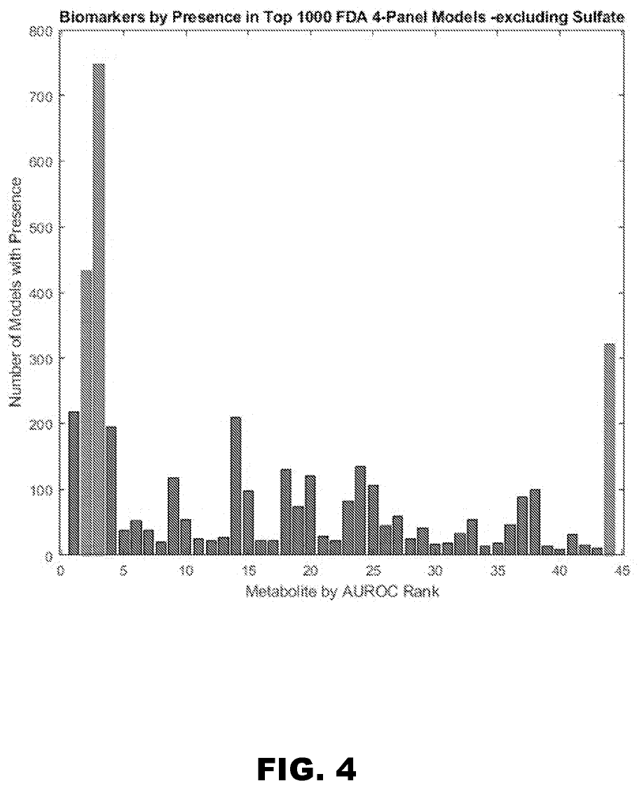

The majority of the top-1000 performing FDA models tended to share the same markers of interest. All three of the markers observed to constitute the optimized 3-variable model (free sulfate, uridine and beta-amino isobutyrate) were also found in all other optimized models as well (FIG. 3). Given the relatively high AUROC ascribed to free sulfate in plasma and total sulfate in plasma, FDA assessments that included these two metabolites as part of the model discovery process tended to skew towards the inclusion of these metabolites in biomarker panels. In order to carry out a more thorough assessment of the remaining significant metabolites, the two sulfate measures were excluded to examine the efficacy of panels consisting of other potential biomarkers. An exhaustive analysis of all possible remaining 4-variable model panels, with leave-one-out cross validation was performed to determine the biomarkers which occurred most frequently in the top 1000 models (FIG. 4).

### Example 4. SVM Models of the Metabolites Distinguish Between ASD and TD Cohorts

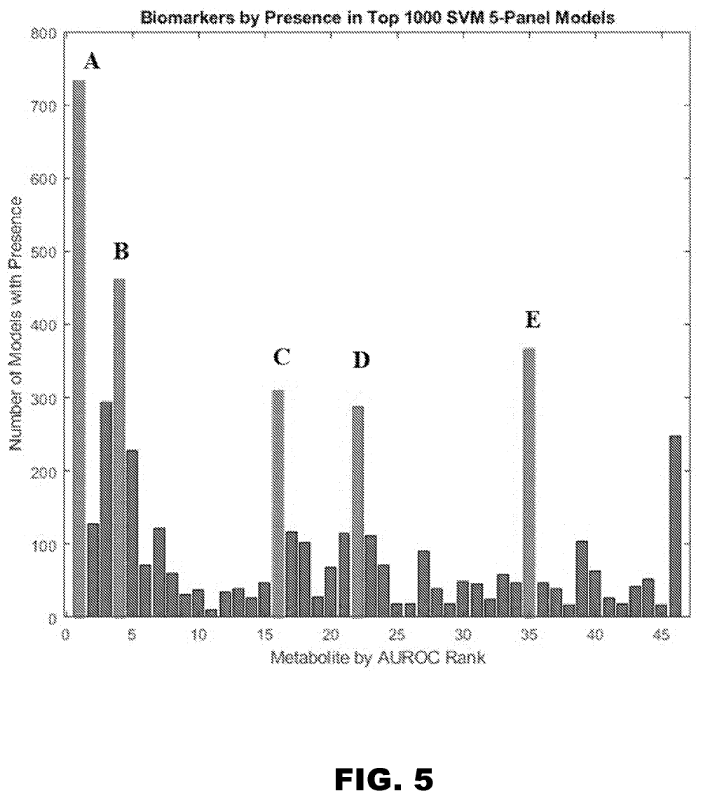

Using an exhaustive classification approach, SVM was used to determine biomarker panels that were best able to distinguish between ASD and TD cohorts. All possible 4-variable panels were determined. This analysis demonstrated the prominence of a few key measured quantities that demonstrated consistent utilization in top performing predictive models. Specifically, free sulfate, glutathione, beta-amino isobutyrate and uridine appeared in more than 20% of all top 1000 performing SVM panels ranked by their cross-validated accuracy (FIG. 5), similar to the results for the FDA models.

### Example 5. Metabolite Correlations Distinguish Between ASD and TD Cohorts

Metabolite Clusters. Table 3 shows the metabolites that were correlated with the top 5 metabolites. Free sulfate was correlated with 11 other metabolites, homocysteine+homocysteine was correlated with 3, uridine was correlated with 2, but beta-amino isobutyrate and magnesium were not correlated with any others. This suggests that the network of significant metabolites correlated with free sulfate represents a major area of metabolic differences between ASD and TD, generally consistent with FIG. 2 which shows most metabolites networked to the sulfate cluster.

TABLE 3
Correlations of top 5 FDA Optimized Variables (bold) with
other measurements, for the ASD group. For all relationships
that met the inclusion criteria below see Tables 4-5.

| | Pearson Correlation |
|---|---|
| Metabolite Pair | Coefficient |
| Free sulfate (plasma) | |
| Total sulfate (plasma) | 0.63 |
| GABA | 0.57 |
| SamR | 0.57 |
| Glutathione | 0.56 |
| Acetylcholine | 0.53 |
| NADH | 0.47 |
| Initial Lithium | 0.45 |
| SAM/SAH | 0.42 |
| Initial Thallium | 0.41 |
| Epinephrine | 0.40 |
| Oxidized Glutathione/ | −0.43 |
| Glutathione | |
| Uridine (plasma) | |
| FIGLU | 0.46 |
| Total sulfate (plasma) | −0.48 |
| Homocysteine + homocystine | |
| Iron | 0.46 |
| Cadmium- (Whole blood) | −0.45 |
| Taurine | −0.55 |
| Beta-amino isobutyrate **** | |
| Magnesium **** | |

Only element pairs with coefficient greater than 0.4 or less than −0.4 are shown.
**** indicates no significant correlations among top ranked metabolites.

TABLE 4
Correlations of top metabolites, xenobiotics and
elements for the ASD group with a Pearson correlation
coefficient greater in magnitude than 0.40

| | | Pearson |
|---|---|---|
| | | Correlation |
| Measurement 1 | Measurement 2 | Coefficient |
| Cadmium | Calcium | 0.49 |
| Iron (Whole blood) | Calcium | −0.89 |
| Taurine | Calcium | 0.45 |
| Cadmium (Whole Blood) | Calcium | 0.47 |
| Cadmium | Iron (Whole blood) | −0.50 |
| Taurine | Iron (Whole blood) | −0.56 |
| Homocysteine + homocystine | Iron (Whole blood) | 0.46 |
| Cadmium | Phosphorus | −0.44 |
| Calcium | Phosphorus | −0.74 |
| Iron (Whole blood) | Phosphorus | 0.71 |
| Taurine | Phosphorus | −0.51 |
| Cadmium (Whole Blood) | Phosphorus | −0.50 |
| Homocysteine + homocystine | Taurine | −0.55 |
| Adenosine (Plasma) | Acetylcholine | −0.51 |
| Free sulfate (plasma) | Acetylcholine | 0.53 |
| GABU | Acetylcholine | 0.60 |
| GSSG/GSH Ratio | Acetylcholine | −0.47 |
| NADP | Acetylcholine | 0.44 |
| Nitrotyrosine | Acetylcholine | −0.54 |
| SamR | Acetylcholine | 0.43 |
| SAM/SAH | Adenosine (Plasma) | −0.45 |
| Glutathione | ATP | 0.44 |
| NADH | ATP | 0.67 |
| NADP | ATP | 0.61 |
| Total Sulfate (plasma) | ATP | 0.44 |
| Cadmium | Cadmium (Whole Blood) | 0.40 |
| Iron (Whole blood) | Cadmium (Whole Blood) | −0.55 |
| Taurine | Cadmium (Whole Blood) | 0.40 |
| Homocysteine + homocystine | Cadmium (Whole Blood) | −0.45 |
| Total Carotenoids | Cadmium (Whole Blood) | −0.46 |
| Copper (RBC) | Copper (whole blood) | 0.46 |
| GSSG/GSH Ratio | Epinephrine | −0.44 |
| Uridine (Plasma) | FIGLU | 0.46 |
| Total Carnitine (carnitine + | Free Carnitine | 0.94 |
| acetyl-carnitine) | | |
| Lithium | Free Sulfate (plasma) | 0.45 |
| Thallium | Free Sulfate (plasma) | 0.41 |
| Epinephrine | Free sulfate (plasma) | 0.40 |
| GABU | Free sulfate (plasma) | 0.57 |
| GSSG/GSH Ratio | Free sulfate (plasma) | −0.43 |
| SAM/SAH | Free sulfate (plasma) | 0.42 |
| SamR | Free sulfate (plasma) | 0.57 |
| Epinephrine | GABU | 0.45 |
| SamR | GABU | 0.47 |
| Acetylcholine | Glutathione | 0.52 |
| Epinephrine | Glutathione | 0.48 |
| Free sulfate (plasma) | Glutathione | 0.56 |
| GABU | Glutathione | 0.52 |
| GSSG/GSH Ratio | Glutathione | −0.63 |
| NADH | Glutathione | 0.67 |
| NADP | Glutathione | 0.47 |
| SamR | Glutathione | 0.50 |
| Total Sulfate (plasma) | Glutathione | 0.60 |
| Acetylcholine | NADH | 0.52 |
| Epinephrine | NADH | 0.56 |
| Free sulfate (plasma) | NADH | 0.47 |
| GABU | NADH | 0.41 |
| GSSG/GSH Ratio | NADH | −0.52 |
| NADP | NADH | 0.66 |
| Nitrotyrosine | NADH | −0.51 |
| Total Sulfate (plasma) | NADH | 0.42 |
| Choline (Total) | NADP | −0.50 |
| GSSG/GSH Ratio | NADP | −0.46 |
| Nitrotyrosine | NADP | −0.45 |
| SamR | NADP | 0.48 |
| GSSG/GSH Ratio | Nitrotyrosine | 0.46 |
| SamR | Nitrotyrosine | −0.44 |
| GSSG/GSH Ratio | Oxidized glutathione | 0.93 |
| Nitrotyrosine | Oxidized glutathione | 0.43 |
| Calcium | Potassium | −0.63 |
| Iron (Whole blood) | Potassium | 0.55 |
| Phosphorus | Potassium | 0.77 |
| Acetylcholine | Total Sulfate (plasma) | 0.57 |
| Free sulfate (plasma) | Total Sulfate (plasma) | 0.64 |
| GABU | Total Sulfate (plasma) | 0.56 |
| GSSG/GSH Ratio | Total Sulfate (plasma) | −0.42 |
| NADP | Total Sulfate (plasma) | 0.52 |
| SamR | Total Sulfate (plasma) | 0.59 |
| Uridine (Plasma) | Total Sulfate (plasma) | −0.48 |

TABLE 5
Correlations of top metabolites, xenobiotics and
elements for the TD group with a Pearson correlation
coefficient greater in magnitude than 0.40.

| | | Pearson |
|---|---|---|
| | | Correlation |
| Measurement 1 | Measurement 2 | Coefficient |
| Glutathione | NADH | 0.48 |
| Glutathione | NADP | 0.49 |
| Glutathione | Epinephrine | 0.41 |
| Glutathione | Norepinephrine | 0.51 |
| Glutathione | Serotonin | 0.45 |
| Glutathione | Acetylcholine | 0.45 |
| Glutathione | Sulfate (Total) | 0.55 |
| Glutathione | GABU | 0.49 |
| Glutathione | SAM/SAH | 0.45 |
| ATP | GSSG/GSH Ratio | −0.42 |
| ATP | NADH | 0.50 |
| ATP | NADP | 0.67 |
| ATP | Sulfate (Free) | 0.58 |
| ATP | AdeP | −0.42 |
| ATP | Uridine | −0.47 |
| ATP | SAM/SAH | 0.46 |
| ATP | Cadmium | −0.53 |
| Figl | NADH | −0.44 |
| Figl | NADP | −0.50 |
| Figl | Norepinephrine | −0.43 |
| Nitrotyrosine | Oxidized glutathione | 0.56 |
| Nitrotyrosine | GSSG/GSH Ratio | 0.75 |
| Nitrotyrosine | NADP | −0.51 |
| Nitrotyrosine | Choline (Total) | 0.66 |
| Nitrotyrosine | Epinephrine | −0.46 |
| Nitrotyrosine | Norepinephrine | −0.47 |
| Nitrotyrosine | Serotonin | −0.43 |
| Nitrotyrosine | Acetylcholine | −0.72 |
| Nitrotyrosine | Sulfate (Total) | −0.64 |
| Nitrotyrosine | Sulfate (Free) | −0.51 |
| Nitrotyrosine | AdeP | 0.60 |
| Nitrotyrosine | Uridine | 0.46 |
| Nitrotyrosine | GABU | −0.59 |
| Nitrotyrosine | SamR | −0.54 |
| Nitrotyrosine | SAM/SAH | −0.61 |
| Oxidized glutathione | GSSG/GSH Ratio | 0.89 |
| Oxidized glutathione | Choline (Total) | 0.46 |
| Oxidized glutathione | Epinephrine | −0.47 |
| Oxidized glutathione | Acetylcholine | −0.51 |
| GSSG/GSH Ratio | NADH | −0.41 |
| GSSG/GSH Ratio | NADP | −0.50 |
| GSSG/GSH Ratio | Choline (Total) | 0.57 |
| GSSG/GSH Ratio | Epinephrine | −0.57 |
| GSSG/GSH Ratio | Norepinephrine | −0.42 |
| GSSG/GSH Ratio | Serotonin | −0.48 |
| GSSG/GSH Ratio | Acetylcholine | −0.65 |
| GSSG/GSH Ratio | Sulfate (Total) | −0.58 |
| GSSG/GSH Ratio | Sulfate (Free) | −0.46 |
| GSSG/GSH Ratio | Uridine | 0.43 |
| GSSG/GSH Ratio | GABU | −0.56 |
| GSSG/GSH Ratio | SAM/SAH | −0.51 |
| NADH | NADP | 0.81 |
| NADH | Choline (Total) | −0.45 |
| NADH | Norepinephrine | 0.57 |
| NADH | Serotonin | 0.41 |
| NADH | Acetylcholine | 0.50 |
| NADH | Sulfate (Total) | 0.59 |
| NADH | Sulfate (Free) | 0.66 |
| NADH | GABU | 0.50 |
| NADH | SAM/SAH | 0.42 |
| NADP | Choline (Total) | −0.47 |
| NADP | Epinephrine | 0.46 |
| NADP | Norepinephrine | 0.61 |
| NADP | Serotonin | 0.55 |
| NADP | Acetylcholine | 0.46 |
| NADP | Sulfate (Total) | 0.62 |
| NADP | Sulfate (Free) | 0.66 |
| NADP | AdeP | −0.41 |
| NADP | Uridine | −0.48 |
| NADP | GABU | 0.60 |
| NADP | SamR | 0.53 |
| NADP | SAM/SAH | 0.50 |
| Choline (Total) | Norepinephrine | −0.49 |
| Choline (Total) | Acetylcholine | −0.64 |
| Choline (Total) | Sulfate (Total) | −0.52 |
| Choline (Total) | Sulfate (Free) | −0.41 |
| Choline (Total) | AdeP | 0.49 |
| Choline (Total) | SamR | −0.49 |
| Choline (Total) | Taurine | −0.53 |
| Free Carnitine | Total Carnitine (carnitine + | 0.97 |
| | acetyl-carnitine) | |
| Total Carnitine (carnitine + | Vitamin B5 | −0.42 |
| acetyl-carnitine) | | |
| Total Carnitine (carnitine + | Serine | 0.48 |
| acetyl-carnitine) | | |
| Epinephrine | Norepinephrine | 0.57 |
| Epinephrine | Serotonin | 0.59 |
| Epinephrine | Acetylcholine | 0.53 |
| Epinephrine | Sulfate (Total) | 0.71 |
| Epinephrine | Sulfate (Free) | 0.44 |
| Epinephrine | GABU | 0.66 |
| Norepinephrine | Serotonin | 0.59 |
| Norepinephrine | Acetylcholine | 0.49 |
| Norepinephrine | Sulfate (Total) | 0.70 |
| Norepinephrine | GABU | 0.74 |
| Norepinephrine | SamR | 0.46 |
| Serotonin | Acetylcholine | 0.43 |
| Serotonin | Sulfate (Total) | 0.75 |
| Serotonin | Sulfate (Free) | 0.51 |
| Serotonin | GABU | 0.62 |
| Acetylcholine | Sulfate (Total) | 0.69 |
| Acetylcholine | Sulfate (Free) | 0.54 |
| Acetylcholine | Adenosine | −0.54 |
| Acetylcholine | GABU | 0.61 |
| Acetylcholine | SamR | 0.59 |
| Acetylcholine | SAM/SAH | 0.50 |
| Sulfate (Total) | Sulfate (Free) | 0.65 |
| Sulfate (Total) | GABU | 0.70 |
| Sulfate (Total) | SamR | 0.49 |
| Sulfate (Total) | SAM/SAH | 0.53 |
| Sulfate (Total) | Lithium | 0.45 |
| Sulfate (Free) | Adenosine | −0.41 |
| Sulfate (Free) | GABU | 0.51 |
| Sulfate (Free) | SAM/SAH | 0.49 |
| VDC | Tin | 0.40 |
| Biotin | Cadmium (Whole Blood) | 0.58 |
| Biotin | Calcium | 0.52 |
| Biotin | Iron (RBC) | −0.46 |
| Biotin | Thallium | −0.46 |
| Biotin | Taurine | 0.46 |
| Biotin | Glutamate | 0.45 |
| Biotin | Vitamin B5 | 0.66 |
| Adenosine | SamR | −0.53 |
| Uridine | SAM/SAH | −0.58 |
| GABU | SamR | 0.44 |
| SamR | SAM/SAH | 0.71 |
| Copper (WB) | Copper (RBC) | 0.70 |
| Cadmium (Whole Blood) | Calcium | 0.64 |
| Cadmium (Whole Blood) | Iron (RBC) | −0.56 |
| Calcium | Potassium | −0.47 |
| Calcium | Phosphorus | −0.59 |
| Calcium | Iron (RBC) | −0.76 |
| Potassium | Phosphorus | 0.71 |
| Potassium | Iron (RBC) | 0.50 |
| Potassium | Taurine | −0.40 |
| Phosphorus | Iron (RBC) | 0.65 |
| Iron (RBC) | Taurine | −0.48 |
| Iron (RBC) | Taurine | −0.48 |
| Magnesium | Glutamate | −0.42 |
| Taurine | Homocysteine + homocystine | −0.41 |
| Tryptophan | Vitamin B5 | 0.43 |

TABLE 6
2-Variable FDA models that achieved the highest AUROC
following cross-validation (CV). CV AUROC was calculated
by using leave-one-out cross-validation

| | | Raw | CV |
|---|---|---|---|
| Norepinephrine | Compound 2 | AUROC | AUROC |
| Free Sulfate | Uridine (Plasma) | 0.924 | 0.938 |
| Free Sulfate | Beta-amino isobutyrate | 0.939 | 0.934 |
| Free Sulfate | Homocysteine + | 0.916 | 0.933 |
| | Homocystine | | |
| Free Sulfate | Thallium | 0.920 | 0.924 |
| Total Sulfate | Uridine (Plasma) | 0.898 | 0.918 |
| Free Sulfate | Phosphorus | 0.909 | 0.911 |
| Free Sulfate | VDC | 0.910 | 0.910 |
| Free Sulfate | Cadmium | 0.910 | 0.907 |
| Total Sulfate | Iron | 0.902 | 0.906 |
| Free Sulfate | Glutathioneamate | 0.905 | 0.906 |
| Uridine (Plasma) | Homo cystine | 0.908 | 0.905 |
| Free Sulfate | Tin | 0.905 | 0.905 |
| Free Sulfate | Lead | 0.905 | 0.905 |
| Serotonin | Free Sulfate | 0.894 | 0.902 |
| Free Sulfate | Copper | 0.904 | 0.900 |
| Glutathione | Free Sulfate | 0.898 | 0.899 |
| Nitrotyrosine | Free Sulfate | 0.900 | 0.899 |
| Free Sulfate | Cadmium | 0.900 | 0.898 |
| Free Sulfate | Lithium | 0.898 | 0.896 |
| Total Sulfate | Homocysteine + | 0.877 | 0.895 |
| | Homocystine | | |
| Nitrotyrosine | Total Sulfate | 0.876 | 0.895 |
| Free Sulfate | Serine | 0.897 | 0.895 |
| Glutathione | Thallium | 0.889 | 0.894 |
| Free Sulfate | SAM/SAH | 0.893 | 0.894 |
| Total Sulfate | Beta-amino isobutyrate | 0.884 | 0.893 |
| Glutathione | Uridine (Plasma) | 0.888 | 0.893 |
| Free Sulfate | Biotin | 0.906 | 0.891 |
| Oxgl/Glutathione | Free Sulfate | 0.891 | 0.890 |
| NADH | Total Sulfate | 0.876 | 0.890 |
| Total Sulfate | VDC | 0.881 | 0.890 |
| Total Sulfate | Cadmium | 0.879 | 0.890 |
| Total Sulfate | Tryptophan | 0.874 | 0.890 |
| Free Sulfate | GABU | 0.894 | 0.890 |
| Nitrotyrosine | Homocysteine + | 0.895 | 0.888 |
| | Homocystine | | |
| ChoT | Free Sulfate | 0.890 | 0.888 |
| Free Sulfate | Tryptophan | 0.903 | 0.888 |
| Total Sulfate | Phosphorus | 0.878 | 0.887 |
| NADH | Uridine (Plasma) | 0.888 | 0.887 |
| Total Sulfate | Tin | 0.858 | 0.886 |
| Nitrotyrosine | Uridine (Plasma) | 0.887 | 0.886 |
| Nitrotyrosine | Glutathioneamate | 0.885 | 0.886 |
| Free Sulfate | Taurine | 0.896 | 0.886 |
| ATP | Free Sulfate | 0.892 | 0.884 |
| Oxidized glutathione | Free Sulfate | 0.892 | 0.883 |
| Free Sulfate | VDTC | 0.890 | 0.883 |
| Epinephrine | Free Sulfate | 0.893 | 0.883 |
| Glutathione | Homocysteine + | 0.871 | 0.882 |
| | Homocystine | | |
| NADP | Free Sulfate | 0.893 | 0.881 |
| Norepinephrine | Uridine (Plasma) | 0.878 | 0.881 |
| Free Sulfate | Calcium | 0.897 | 0.881 |

### Discussion

The use of multivariate analysis allows for a more comprehensive evaluation to distinguish ASD and TD cohorts using a biochemical approach. Furthermore, investigating the nature of interactions and relationships among metabolites that significantly differ between ASD and TD cohorts provides insight into how cellular processes and environmental factors may have different influences between such groups.

Univariate Findings. The use of FDR to account for multiple hypothesis testing revealed 46 variables that were statistically significant, more than was previously identified using different statistical methods. Many of the metabolites that were statistically significant have shown to be prominent in processes related to oxidative stress, methylation, sulfation, and mitochondrial metabolism. Overall, five metabolites were primary amino acids, eight were related to oxidative stress, eleven were nutrients/vitamins, and five were neurotransmitters. While most identified compounds were related to biological systems and metabolism, four toxicants were also identified as having a significantly different levels between the ASD and TD groups of children. Nonetheless, as the concentration of xenobiotics were derived from urinary measurements, this does not necessarily reflect a higher total body prevalence.

Metabolites associated with the FOCM/TS pathways were found to be both statistically significant and have high AUROC values, which ranged from 0.65-0.85. Glutathione, SAM/SAH ratio and oxidized glutathione were all found to be significantly distinct between both cohorts, and all are related to impaired methylation. The metabolic co-factors ATP. NADP, and NADH in plasma were identified as having higher AUROC values relative to most other metabolites examined. All three were observed to have an AUROC greater than 0.70 and to be significantly lower in the ASD cohort.

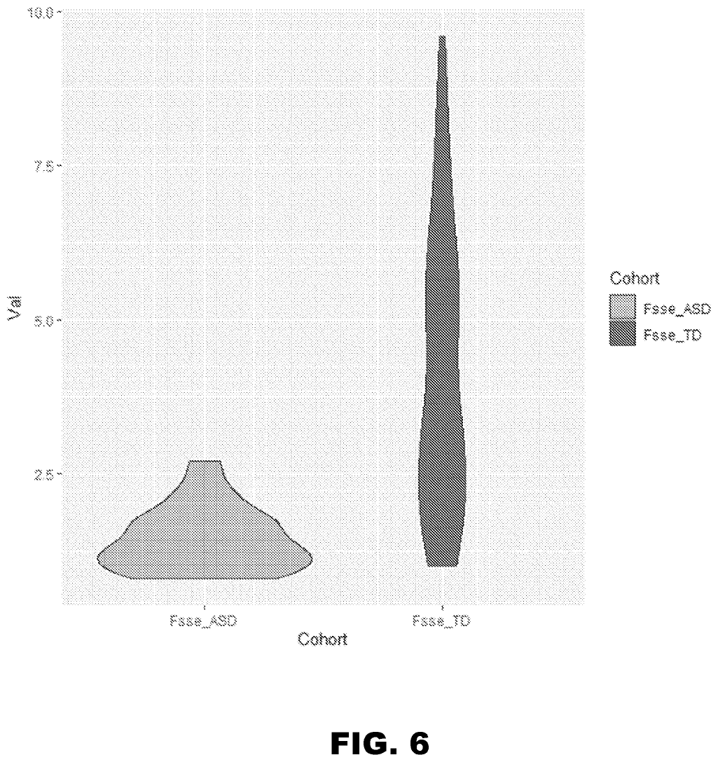

Total and free sulfate were identified as being especially prominent metabolites in terms of their statistical significance between the ASD and TD cohorts (FIG. 6). A significant body of work has shown that significant differences in sulfation capacity and sulfur related metabolites have been commonly observed between ASD and TD cohorts. Urinary elemental sulfur concentrations were found to be significantly lower in children with ASD, and were a prominent contributor to FDA models for distinguishing between ASD and TD groups. Sulfate metabolism is closely connected to interactions of the gut microbiome, and the presence of certain organic sulfate compounds have been statistically higher in feces of children with ASD.

Four neurotransmitters (serotonin, norepinephrine, epinephrine, and acetylcholine) were measured in platelets and found be to significantly lower in the ASD group. Platelet serotonin receptor binding among children with ASD has commonly been reported as being lower when compared to typically developing controls. These abnormalities are likely contributing to some of the neurological and behavioral symptoms of ASD. In contrast, glutamate (measured in plasma) was found to be significantly higher in the ASD group, and GABA (in urine) was found to be significantly lower. Glutamate is the primary excitatory neurotransmitter, and GABA is the primary inhibitory neurotransmitter, so the increased ratio of glutamate:GABA likely contributes to certain autism symptoms including seizures, repetitive behaviors, and difficulty regulating emotions.

Levels of l-carnitine, acetyl-I-carnitine, and their sum (total carnitine) were found to be significantly higher in the ASD group. These results are consistent with previous findings that children with ASD may have a decreased ability to conjugate carnitine and that carnitine supplementation was beneficial to children with ASD.

Correlation Analyses. Correlation analysis was performed to provide insight into the relationships between the significant measurement variables. The ASD group had many fewer correlation pairs than the TD group (106 vs 189), suggesting disruption of many metabolic processes (FIG. 1-2). Differences in metabolomic relationships may indicate areas of divergence of underlying processes, and metabolic pathway differences has been a frequent subject of research regarding ASD etiology.

The metabolites that appeared most frequently in the optimized FDA multivariate predictive models were free total sulfate, beta-amino isobutyrate, homocysteine-homocystine, magnesium and uridine (Table 3). The number of unique correlations observed among these measurements followed similar trends as other significant metabolites in that there were a greater number of correlations corresponding to the TD cohort when compared to that of the ASD group. Free total sulfate had the greatest number of significant correlations, with 11 relationship pairs among other significant metabolites for the ASD group and 18 for the TD group (Table 1). As a product of the transulfuration pathway, several FOCM/TS related metabolites such as SAM/SAH, glutathione and total sulfate were significantly correlated as well. Uridine was found to be correlated with FIGLU which is known to be an indicator for methylation insufficiency. Given the nature of FDA, relationships using orthogonal variables work best for distinguishing groups. Subsequently, two metabolites with limited correlations to others were utilized for multi-variate classification analysis (beta-amino isobutyrate and magnesium), and may represent other areas of metabolic differences.

Metabolites associated with neurotransmitters were found to have a much higher number of correlations in the TD group than the ASD group. The neurotransmitter serotonin also contrasted prominently between cohorts. The TD group was observed to have 9 metabolites correlated with serotonin, but only magnesium was significantly correlated with serotonin in the ASD cohort (FIGS. 1-2). Notably, for the ASD cohort, no significant correlation was observed between serotonin and its amino acid precursor tryptophan.

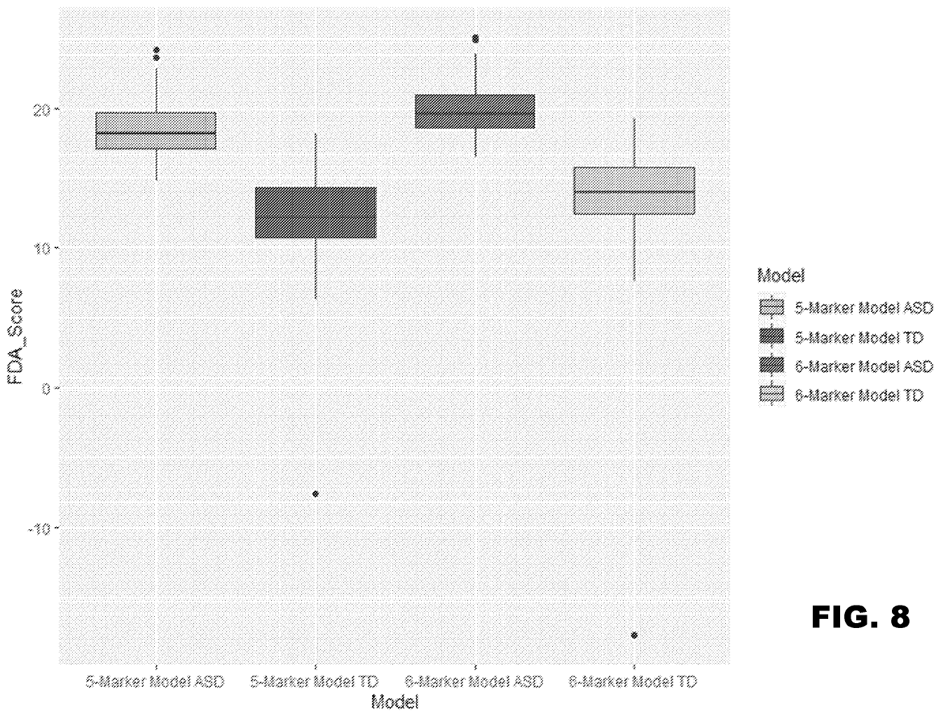

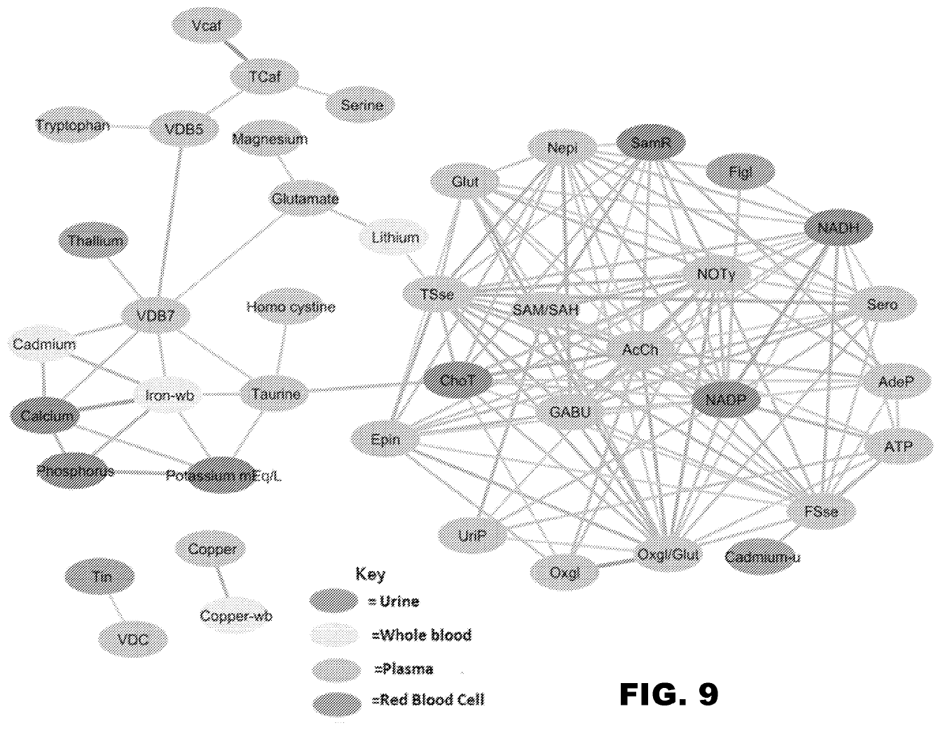

The relationships for a number of B vitamins were found to be distinct between the ASD and TD cohorts. Pantothenic acid (vitamin B5) was observed to have 3 significant correlations in the TD cohort but was not found to have any such relationships in the ASD group (FIG. 1, FIG. 8-9). Tryptophan was observed to be significantly correlated with pantothenic acid for the TD cohort but was not found to have any such relationship for the ASD group (FIG. 1-2).

The relationship between ASD symptom severity and metabolomics has been an area of considerable investigation. The initial findings from this study demonstrated that there were several metabolites correlated with ASD behavioral symptom severity. Free sulfate in plasma was the only metabolite found to be significantly correlated (negatively) with SAS score (r=−0.38). However, free sulfate was in turn highly correlated with eight other significant metabolites in the ASD group. It was also observed that iron found in red blood cells was the sole metabolite significantly correlated with behavioral symptoms as surmised by the PDD-BI score (r=0.36). The results suggested that ASD severity was associated with a wide number of metabolic and nutritional differences.

Multivariate Analysis for Classifying ASD. Using the comprehensive data collected on biochemical compounds examined in this work, ASD characterization leveraging this metabolomic data was explored. As ASD is only formally diagnosed through psychometric evaluation, development of a biochemical test would have considerable promise for supporting this process and potentially providing an avenue for earlier diagnosis. While the average age of ASD diagnosis in the United States is 51 months, stable diagnosis has been ascertained as early as 14 months. Consequently, a biochemical test supporting a diagnosis may lead to earlier intervention and treatment such as Applied Behavioral Analysis.

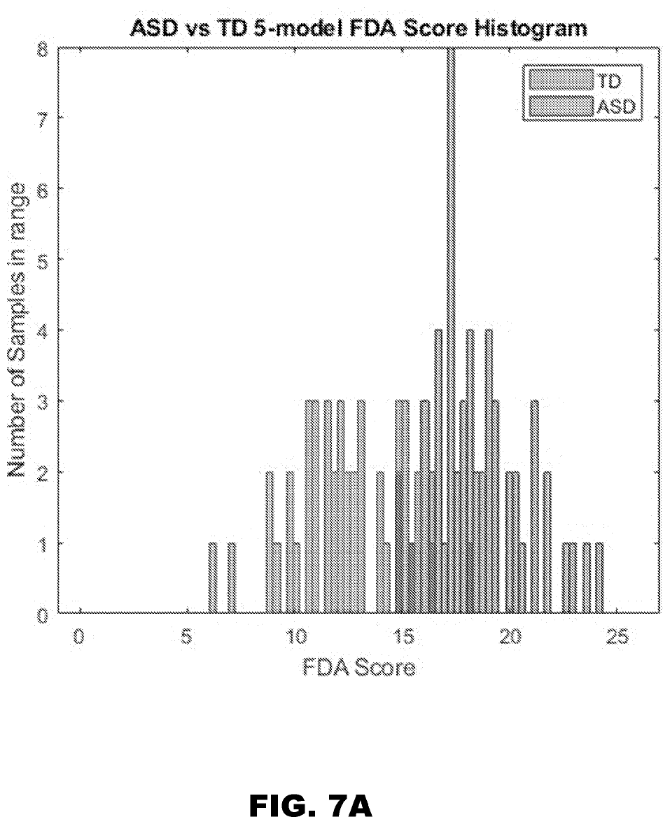

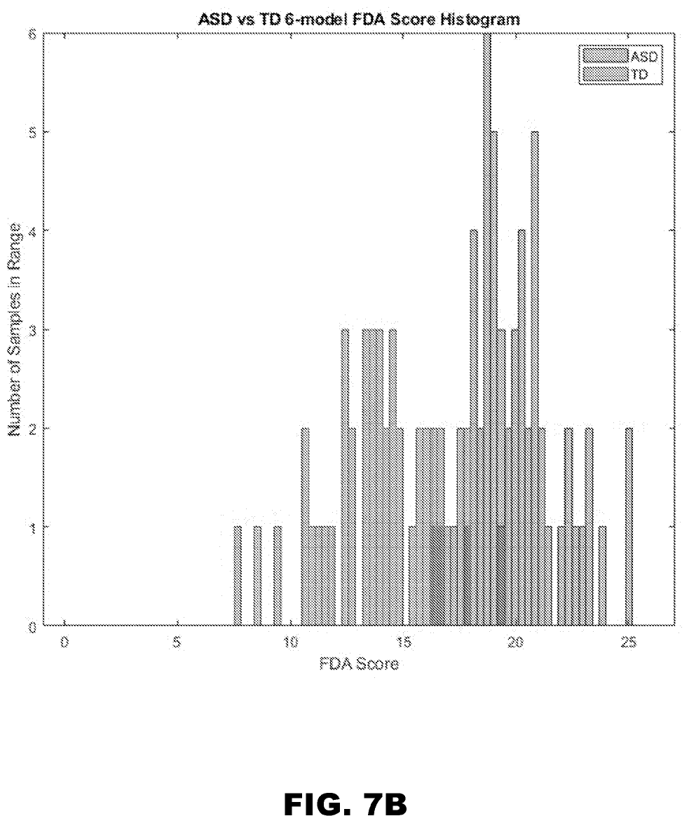

Multivariate analysis using significant measured variables outperformed all individual univariate assessments for classification between the ASD and TD groups. Using both the entire dataset as well as only those with complete sets of measurements, it was possible to attain models with a cross-validation accuracy greater than 0.96 (FIGS. 7A, 7B, and 8). The composition of the models with three or more components that were able to achieve the highest accuracy were consistently composed of free sulfate, uridine and beta-amino isobutyrate. Despite a relatively high AUROC, total sulfate did not appear prominently in the FDA model panels developed because of its high degree of correlation with free sulfate. When omitting sulfate metabolites from the FDA model discovery protocol, relevant models consisting of sulfate-correlated metabolites were more common. Uridine was still often selected, appearing in over 74.7% of the top 1000 models. In the sulfur-excluding models, plasma glutathione appeared in 47.1% of all models while, plasma homocystine was present in 32.1% and plasma nitro-tyrosine in 21.8% of models.

Beta-amino isobutyrate had a high AUROC value (0.69) and was identified prominently in all top performing FDA models with the inclusion of sulfate-based metabolites. A product of thymine catabolism, the circulating levels of this metabolite are controlled by alanine: glyoxylate aminotransferase 2, which is a mitochondrial enzyme. As a prominent antioxidant, glutathione plays a crucial role in several cellular processes. It is responsible for cellular signaling, detoxification and responding to oxidative stress.

In this study, while the cross-validated accuracy using SVM models were slightly lower compared to the results of the top FDA models (0.92 vs 0.98), the constituents of the model panels that achieved the highest cross-validated accuracy were largely in concordance (Table 2). Free sulfate in plasma was the top reported metabolite prevalent in models, appearing in 74.6% of the top 1,000 models. Similarly, both glutathione, beta-amino isobutyrate and uridine appeared in more than 20% of the top models. The accuracy of characterization observed from the SVM analysis as applied in the instant disclosure was better than previous attempts at distinguishing between ASD and TD groups using biochemical measurements.

### CONCLUSION

Models consisting of free sulfate in plasma, plasma uridine, and beta-amino isobutyrate achieved the highest AUROC after applying leave-one-out cross-validation using the full dataset of 99 individuals with both SVM and FDA techniques. Models consisting of these metabolites achieved a fitted AUROC of 0.98 for FDA and 0.92 for SVM. The highest univariate AUROC value was observed for free sulfate in plasma, which was a biomarker in all optimized top 5+ marker panels. It is important to note however that non-sulfate containing models were able to achieve similar results because many other measurements are correlated with sulfate. Overall, the results using statistical classification techniques for ASD diagnosis prediction resulted in a cross-validated performance that is among the highest compared to prior panels investigated in the literature.

The disclosure proved herein examined the degree of interconnectivity of statistically significant variables amongst themselves contrasted between the ASD and TD group. In general, the ASD cohort had a much lower number of correlations between metabolites, suggesting a disruption of many metabolic processes. Supplementation with vitamins/minerals/micronutrients has been demonstrated to normalize many metabolic pathways and improve some ASD-related symptoms.

### SUMMARY

Provided herein is a comparative study contrasting metabolomic and nutrient measurements of children with ASD (n=55) against their typically developing (TD) peers (n=44) through a multivariate statistical lens. Hypothesis testing, receiver characteristic curve assessment and correlation analysis served to underscore prominent areas where metabolomic and nutritional profiles between the groups diverged. Improved univariate analysis revealed 46 nutritional/metabolic differences being significantly different between ASD and TD groups, with individual AUROC scores of 0.6-0.9. Many of the significant measurements had correlations with many others, forming two integrated networks of inter-related metabolic differences in ASD. The TD group had 189 significant correlation pairs between metabolites, vs only 106 for the ASD group, calling attention to underlying differences in metabolic processes. Furthermore, multivariate techniques identified potential biomarker panels with up to six metabolites that were able to attain a predictive accuracy of up to 98% for discriminating between ASD and TD, following cross-validation. Assessing all optimized multivariate models demonstrated concordance with prior physiological pathways identified in the literature, with some of the most important metabolites for discriminating ASD and TD being sulfate, uridine (methylation biomarker) and beta-amino isobutyrate.

TABLE 7
Combinations of two compounds identified using FDA models that
achieved the highest AUROC following cross-validation (CV). CV
AUROC was calculated by using leave-one-out cross-validation.

| | | Raw | CV |
|---|---|---|---|
| Compound 1 | Compound 2 | AUROC | AUROC |
| Free Sulfate | Uridine (Plasma) | 0.924 | 0.938 |
| Free Sulfate | Beta-amino isobutyrate | 0.939 | 0.934 |
| Free Sulfate | Homocysteine + | 0.916 | 0.933 |
| | Homocystine | | |
| Free Sulfate | Thallium | 0.920 | 0.924 |
| Total Sulfate | Uridine (Plasma) | 0.898 | 0.918 |
| Free Sulfate | Phosphorus | 0.909 | 0.911 |
| Free Sulfate | VDC | 0.910 | 0.910 |
| Free Sulfate | Cadmium | 0.910 | 0.907 |
| Total Sulfate | Iron | 0.902 | 0.906 |
| Free Sulfate | Glutathioneamate | 0.905 | 0.906 |
| Uridine (Plasma) | Homo cystine | 0.908 | 0.905 |
| Free Sulfate | Tin | 0.905 | 0.905 |
| Free Sulfate | Lead | 0.905 | 0.905 |
| Serotonin | Free Sulfate | 0.894 | 0.902 |
| Free Sulfate | Copper | 0.904 | 0.900 |
| Glutathione | Free Sulfate | 0.898 | 0.899 |
| Nitrotyrosine | Free Sulfate | 0.900 | 0.899 |
| Free Sulfate | Cadmium | 0.900 | 0.898 |
| Free Sulfate | Lithium | 0.898 | 0.896 |
| Total Sulfate | Homocysteine + | 0.877 | 0.895 |
| | Homocystine | | |
| Nitrotyrosine | Total Sulfate | 0.876 | 0.895 |
| Free Sulfate | Serine | 0.897 | 0.895 |
| Glutathione | Thallium | 0.889 | 0.894 |
| Free Sulfate | SAM/SAH | 0.893 | 0.894 |
| Total Sulfate | Beta-amino isobutyrate | 0.884 | 0.893 |
| Glutathione | Uridine (Plasma) | 0.888 | 0.893 |
| Free Sulfate | Biotin | 0.906 | 0.891 |
| Oxgl/Glutathione | Free Sulfate | 0.891 | 0.890 |
| NADH | Total Sulfate | 0.876 | 0.890 |
| Total Sulfate | VDC | 0.881 | 0.890 |
| Total Sulfate | Cadmium | 0.879 | 0.890 |
| Total Sulfate | Tryptophan | 0.874 | 0.890 |
| Free Sulfate | GABU | 0.894 | 0.890 |
| Nitrotyrosine | Homocysteine + | 0.895 | 0.888 |
| | Homocystine | | |
| ChoT | Free Sulfate | 0.890 | 0.888 |
| Free Sulfate | Tryptophan | 0.903 | 0.888 |
| Total Sulfate | Phosphorus | 0.878 | 0.887 |
| NADH | Uridine (Plasma) | 0.888 | 0.887 |
| Total Sulfate | Tin | 0.858 | 0.886 |
| Nitrotyrosine | Uridine (Plasma) | 0.887 | 0.886 |
| Nitrotyrosine | Glutathioneamate | 0.885 | 0.886 |
| Free Sulfate | Taurine | 0.896 | 0.886 |
| ATP | Free Sulfate | 0.892 | 0.884 |
| Oxidized glutathione | Free Sulfate | 0.892 | 0.883 |
| Free Sulfate | VDTC | 0.890 | 0.883 |
| Epinephrine | Free Sulfate | 0.893 | 0.883 |
| Glutathione | Homocysteine + | 0.871 | 0.882 |
| | Homocystine | | |
| NADP | Free Sulfate | 0.893 | 0.881 |
| Norepinephrine | Uridine (Plasma) | 0.878 | 0.881 |
| Free Sulfate | Calcium | 0.897 | 0.881 |

1. A method of identifying Autism Spectrum Disorder (ASD) in a subject suspected of having or at risk of having ASD, the method comprising:
a. measuring or having measured a level of each of two or more metabolites in a combination of at least two metabolites, wherein each of the metabolite is selected from the metabolites listed in Table 1 in a biological sample obtained from the subject;
b. comparing the measured level of each metabolite in the selected combination against a control panel of metabolite levels created by measuring a metabolite level in control typically developing (TD) subjects, wherein the comparing comprises:
i. calculating the sensitivity (TPR) and specificity (TNR) for the combination of metabolites using Fisher Discriminant Analysis (FDA) or support vector machines (SVM); and
ii. calculating the area under the curve (AUC) of the receiver operating characteristic (ROC) curve (AUROC) for the combination of two or more metabolites using the FPR and TPR calculated in (b); or
iii. performing hypotheses testing by evaluating the type of distribution for each of the ASD and TD groups' measurements and selecting an appropriate parametric or non-parametric test determined by a normality and variance of each individual clinical measurement variable for each of the ASD and TD groups; and
c. identifying the subject as having or at risk of ASD when the level of each of the metabolite in the combination in the biological sample is significantly different from the level of the metabolite in the control panel of metabolite levels: when (i) the AUROC value for the combination of metabolites is about 0.85 or higher; or (ii) a significant difference between each of the metabolite in the combination is observed using hypothesis testing.

2. (canceled)

3. (canceled)

4. (canceled)

5. (canceled)

6. The method of claim 1, wherein a parametric test is performed when a normality assumption holds true for the ASD and TD groups.

7. The method of claim 6, wherein an equal variance t-test or Welch's test (unequal variance t-test) is performed if the observed variance is significantly different between the ASD and TD groups.

8. The method of claim 1, wherein a Mann-Whitney test is performed if the ASD and TD groups follow the same non-parametric distribution.

9. The method of claim 8, wherein the ASD and TD groups are adjusted by their means and subjected to the Kolmogorov-Smirnov test where different distributions are observed.

10. The method of claim 1, further comprising determining the false discovery rate (FDR) for each measurement using a leave-one-out (L-1-O) approach.

11. The method of claim 1, wherein the combination of two or more metabolites are identified by performing multivariate analysis using Fisher Discriminant Analysis (FDA) and support vector machines (SVM) and selecting the combinations of two or more metabolites comprising the best FDA or SVM measure for each combination of elements.

12. The method of claim 11, wherein performing FDA comprises:
a. evaluating all possible combinations of two, three, and four-metabolites from among the metabolites of Table 1;
b. examining the fitted AUROC and performance when subjected to cross-validation using leave-one-out cross-validation; and
c. calculating the area under the AUROC value for each combination of two, three, and four-metabolites;
wherein the combination of two or more metabolites comprises the combinations of two, three, and four-metabolites comprising the highest 1000 AUROC values following leave-one-out cross-validation.

13. The method of claim 12, further comprising using a greedy algorithm to identify combinations of metabolites comprising five or more metabolites.

14. The method of claim 12, wherein performing SVM comprises assessing all possible combinations of 5-metabolites and subjecting each combination of five or more metabolites leave-one-out cross-validation if the combination of five or more metabolites attains an accuracy greater than 0.90.

15. The method of claim 1, wherein the biological sample is whole blood, plasma, red blood cells (RBCs), urine, or any combination thereof.

16. The method of claim 1, wherein the combination of two or more metabolites comprises a combination of metabolites of Table 7 and Table 2.

17. (canceled)

18. The method claim 1, wherein the method diagnoses ASD at birth or pre-birth.

19. The method of claim 1, wherein: (a) the level of each of two metabolite in a selected combination of at least two metabolites are measured, wherein the at least two metabolites in combination is selected from Table 7; (b) the level of each of three metabolite in a selected combination of at least three metabolites are measured, wherein the three metabolites in combination is free sulfate, uridine, and beta-amino isobutyrate; (c) the level of each of four metabolite in a selected combination of at least four metabolites are measured, wherein the four metabolites in combination is free sulfate, uridine, homo cystine, and beta-amino isobutyrate; (d) the level of each of five metabolite in a selected combination of at least five metabolites are measured, wherein the five metabolites in combination is selected from free sulfate, uridine, homo cystine, beta-amino isobutyrate, and magnesium; free sulfate, uridine, homo cystine, beta-amino isobutyrate, and tryptophan; and free sulfate, magnesium, homo cystine, uridine, and beta-amino isobutyrate; or the level of each of six metabolite in a selected combination of at least six metabolites are measured, wherein the six metabolites in combination is free sulfate, homo cystine, uridine, beta-amino isobutyrate, magnesium, and copper.

20. (canceled)

21. (canceled)

22. The method of claim 19, wherein the (a) each of the level of the free sulfate, the uridine, homo cystine, tryptophan, and the level of beta-amino isobutyrate is measured in a plasma sample; (b) level of magnesium is measured in a serum sample; and (c) the level of copper is measured in an RBC sample.

23. (canceled)

24. (canceled)

25. (canceled)

26. (canceled)

27. (canceled)

28. (canceled)

29. (canceled)

30. (canceled)

31. (canceled)

32. The method of claim 1, further comprising administering a medical, behavioral, or nutritional treatment to the subject identified as having or at risk of ASD.

33. The method of claim 32, wherein the treatment comprises (a) adjusting the level of one or a combination of two or more metabolites in the subject; or (b) administering a combination of anti-oxidants and a source of sulfate.

34. (canceled)

35. A method of determining a personalized treatment for a subject suspected of having or at risk of having ASD, the method comprising identifying the subject as having or at risk of having ASD using the method of claim 1, and 0 administering a personalized medical, behavioral, or nutritional treatment protocol to the subject.

36. (canceled)

37. A method of monitoring a therapeutic effect of an ASD treatment protocol in a subject suspected of having or at risk of having ASD, the method comprising:
a. measuring in a first biological sample obtained from the subject the level of each of a metabolite in a selected combination of at least two metabolites, wherein each of the metabolite is selected from the metabolites listed in Table 1;
b. measuring in a second biological sample obtained from the subject at a period of time after the first biological sample is obtained the level of the each of the metabolite in the selected combination; and
c. comparing the level of the each of the metabolite in the selected combination in the first sample and the second sample;
wherein maintenance of the level of the one or combination of two or more metabolites or a change of the level of the one or combination of two or more metabolites to a level of the one or combination of two or more metabolites in a control panel of metabolite levels created by measuring metabolite levels of the one or combination of two or more metabolites in control TD subjects is indicative that the treatment protocol is therapeutically effective in the subject.

38. A kit for diagnosing Autism Spectrum Disorder (ASD) in a subject suspected of having or at risk of having ASD, determining a personalized treatment protocol, monitoring a therapeutic effect of an ASD treatment protocol, or any combination thereof, the kit comprising: (a) a container for collecting a biological sample from the subject; (b) reagents for preparing an extract from a biological sample obtained from the subject; and (c) instructions for (i) preparing the extract, (ii) measuring the level of one or more metabolites selected from the metabolites listed in Table 1; and (iii) applying the measured metabolite levels against a control panel of metabolite levels obtained from typically developing (TD) individuals.
